(19) United States
(12) Patent Application Publication
(10) Pub. No.: US 2007/0274997 A1
(43) Pub. Date: Nov. 29, 2007

(54) Compositions and Methods for Herpes Simplex Prophylaxis and Treatment

(76) Inventors: Anthony Simmons, (US); Jianmin Chen, (US)

Correspondence Address: FULBRIGHT & JAWORSKI L.L.P., 600 CONGRESS AVE., SUITE 2400, AUSTIN, TX, 78701, US

(21) Appl. No.: 10/565,415

(22) PCT Filed: Jul. 26, 2004
(86) PCT No.: PCT/US04/24013

§ 371 Date: Feb. 20, 2007

Related U.S. Application Data
(60) Provisional application No. 60/489,984, filed on Jul. 25, 2003.

(51) Int. Cl. A61K 39/42; C12Q 1/70; C07K 16/10

(52) U.S. Cl. 424/159.1; 530/388.3; 435/5

(57) ABSTRACT

The present invention concerns compositions and methods involving polypeptides, such as single chain antibodies, that target microbial antigens. Embodiments of the invention include compositions and methods related to a prophylactic and therapeutic treatments for microbes that can be neutralized prior to infection of a cell. In particular embodiments, microbes against which the present compositions and methods can be implement include those that cause sexually transmitted diseases (STD) and/or those that display on their surface an antigen that can be the target of compositions of the invention.

This application claims priority to U.S. Provisional Patent Application Ser. No. 60/489,984, filed Jul. 25, 2003, which is incorporated herein by reference in its entirety.

### BACKGROUND OF THE INVENTION

1. Field of the Invention

The present invention relates generally to the fields of microbiology and infectious diseases. More particularly, it concerns compositions and methods for prophylactic or therapeutic treatment of infectious microbes, in particular sexually transmitted microbes.

2. Description of Related Art

Herpes simplex virus type 1 (HSV-1) (e.g., GenBank accession number X14112, GI:1944536, incorporated herein by reference) and its close cousin, herpes simplex virus type 2 (HSV-2) (e.g., GenBank accession number NC_{—}001798, GI:9629267, incorporated herein by reference), collectively HSV, cause various benign diseases, such as the common cold sore found near the lips and also genital herpes. Herpes simplex virus can also cause keratoconjunctivitis, with the potential to lead to blindness, and encephalitis. Individuals who are immunosuppressed are especially vulnerable to HSV infection. HSV infections of immunocompromised individuals and neonates can lead to disseminated and life-threatening disease. Unlike many viruses, once an individual is infected with HSV, the virus remains latent in neurons and can be reactivated by stress or immunosuppression and cause recurrent disease. HSV is generally transmitted by contact of a mucosal surface with HSV. One mechanism for transmission of HSV is by sexual transmission.

Sexually transmitted infections are increasing in developing countries, particularly in South and Southeast Asia, where the epidemic is affecting young women of childbearing age. Also in the U.S. and other western societies, heterosexual transmission of HIV is causing an increasing proportion of AIDS cases (Lifson, 1994). These facts emphasize the need for effective means of protection against heterosexual transmission of HIV and other pathogenic microbes such as Herpes Simplex Virus (HSV).

Three types of preventive methods can be used: i) a physical barrier provided, for example, by a condom, ii) a chemical or pharmaceutical barrier provided by an intravaginal or mucosal microbicide, and iii) an immunological barrier provided by mucosal immunity resulting from a prophylactic vaccine (Elias and Heise, 1994).

Since vaccines giving mucosal protection are probably many years away and condoms, although highly effective in preventing infection by sexually transmitted disease (STD) causing microbes, have failed to become generally accepted by males in many parts of the world, protective means are required which are under the control of the woman and can, if necessary, be used without the knowledge or consent of the male partner. Vaginal microbicides would meet this requirement and could not only protect the female's reproductive tract against infectious agents transmitted by the male, but could vice versa protect the male's genital mucosa against possible infectious agents from the female.

Three types of vaginal microbicides have been considered: i) the microbicides which kill free viruses and virus-infected cells on contact before they can infect the mucosal epithelial cells or lymphocytes and monocytes/macrophages in the mucosa, ii) compounds which prevent infection of mucosal cells by free or cell-associated virus. These include polyanionic polysaccharides and related compounds which are inhibitors of virus adsorption but do not kill virus or virus-infected cells at inhibitory concentrations, and iii) compounds which inhibit replication of virus in infected cells and thus stop the infection locally. Such compounds include, for example, reverse transcriptase inhibitors. The two latter types of compounds are non-contraceptive, i.e. they do not kill sperm cells and are therefore advantageous for women who desire conception but require protection against infection. They are generally water-soluble and supposedly have low toxicity for mucosal membranes. On the other hand, they do not have the broad antimicrobial activity of the membrane-disruptive microbicides, many of which kill a variety of agents causing STD in addition to being spermicidal. A number of products which have been licensed and used as vaginal spermicides have been shown in vitro to have a broad activity against sexually transmitted pathogens including HIV. They include for example nonoxynol-9, octoxynol-9, benzalkonium chloride and menfegol which are used in the form of foams, jellies, creams, sponges, foaming tablets, suppositories, and as coating for condoms. (Rosenberg et al., 1993). Their efficacy in vivo has been questioned.

In addition to their in vitro activities there is some evidence of in vivo efficacy against gonococcal and chlamydial infections ( Louv et al., 1988). The microbicidal activity of nonoxynol-9 has been studied both in vitro and in vivo. However, the results of clinical trials have been controversial (Zekeng et al., 1993), but when used frequently or at a high dose nonoxynol-9 may cause vaginal and cervical lesions which could increase the risk of transmission.

Accordingly, there is a need for new contraceptive and non-contraceptive compositions and methods that can be used frequently without adverse effects.

### SUMMARY OF THE INVENTION

The present invention concerns compositions and methods involving polypeptides, such as single chain antibodies, that target microbial antigens. Embodiments of the invention include compositions and methods related to prophylactic and therapeutic treatments for microbes that can be neutralized prior to and/or after infection of a cell. In particular embodiments, microbes against which the present compositions and methods can be implemented include those that cause sexually transmitted diseases (STD) and/or those that display on their surface an antigen that can be the target of compositions of the invention. In certain embodiments, the microbe includes, but is not limited to, viruses such as HSV, human immunodeficiency virus (HIV), Hepatitis B Virus (HepB) and other sexually transmitted viruses, as well as bacteria such as Chlamydia, Gonorrhea, and other sexually transmitted bacteria. It is also contemplated that the term “microbe” may also refer to fungi and yeast. In particular embodiments the sexually transmitted microbe is HSV.

Embodiments of the invention include a single chain antibody. A single chain antibody (scFv) has a light chain variable region (V_{L}) of an antibody operatively coupled to a heavy chain variable region (V_{H}) of an antibody. Single chain antibodies are described in greater detail below. The V_{L} and V_{H} may be operatively coupled by a flexible linker, which in some embodiments is a peptide linker. A peptide linker may include a series of glycine (Gly) and Serine (Ser) residues. In particular embodiments, the linker may be a (Gly₄Ser)₃ peptide linker. In other embodiments the V_{L} and V_{H} regions may be operatively coupled using various synthetic linkers known in the art. Single chain antibodies of the invention are distinguishable from a monoclonal antibody, which is composed of four polypeptide molecules, two of which contain a V_{L}, while the other two contain a V_{H}. The single chain antibody of the invention may be coupled to a second antibody, including but not limited to a monoclonal antibody, a Fab or other antibody fragment, or a second single chain antibody. A single chain antibody or the invention may be a bispecific antibody. In certain aspects the antibodies of the invention may be humanized or chimeric.

The V_{L} and V_{H} regions will typically be derived from an antibody with a particular binding characteristic, e.g., neutralization of a microbe and inhibition of microbial infectivity, such as by binding a particular surface antigen, for example, HSV glycoprotein. Single chain antibodies can be specifically recombinantly engineered by joining a specific V_{L} region with a specific V_{H} region. A scFv may also be identified by screening single chain antibody libraries for binding to a target protein or target molecule. In various embodiment a source antibody for isolating V_{L} and V_{H} will be a monoclonal antibody. The particular nucleic acid sequences encoding for the variable regions may be cloned by standard molecular biology methods, such as RT PCR and/or other recombinant nucleic acid technologies, which are well known to those of skill in the art.

In certain embodiments, the single chain antibody will have a binding affinity, a binding specificity, and/or inhibitory activity for a microbe of interest, e.g., a microbe that causes or may cause a sexually transmitted disease. In particular embodiments, 1, 2, 3, 4, 5, 6, 7, 8, 9, 10 or more single chain antibodies have a binding activity or a binding specificity towards one or more of the same or different HSV glycoproteins. One or more of the single chain antibodies may attenuate, inhibit, neutralize, block, diminish, or abrogate microbial activity, such as infectivity, cell entry, association with a cell membrane and other activities associated with pathogenicity. In various embodiments, single chain antibodies of the invention will bind at least 1, 2, 3 or more of the same or different HSV glycoproteins or other molecules, e.g., carbohydrate or lipids of the viral surface, that are directly or indirectly involved in the mechanism of infectivity, including virus entry, virus function or the like. In particular embodiments one or more single chain antibodies have a binding affinity and/or a binding specificity for HSV glycoprotein D (HSV gD, e.g., GenBank accession numbers, each of which is incorporated herein by reference, CAA32283, GI:59564 (SEQ ID NO:35) and NP_{—}044536, GI: 9629336 (SEQ ID NO:36)). In certain aspects, a single chain antibody of the invention may bind site VII, and/or site Ib of HSV gD. In certain embodiments, a cocktail of 2, 3, 4, 5, 6, 7, 8, 9, 10, or more single chain antibodies with varying degrees and/or kinds of specificity and binding activities are contemplated. In various embodiments, one of the single chain antibodies has an amino acid sequence as set forth in SEQ ID NO:2.

An antibody of the invention may recognize an epitope of 3, 4, 5, 6, 7, 8, 9, 10, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 393 or 394 amino acids, including any values there between, of SEQ ID NO:35 or SEQ ID NO:36, or any mimetic or variant thereof. Preferably an epitope will include at least amino acids 11 to 19 of gD. More preferably the epitope contains at least amino acid 222 to 252 of gD. In other aspects, an antibody of the invention may recognize an epitope of 3, 4, 5, 6, 7, 8, 9, 10, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 350, 400, 450, 500, 550, 600, 650, 700, 750, 800, 850, 900, or 904 amino acids, including any values there between, of SEQ ID NO:33 or SEQ ID NO:34, or any mimetic or variant thereof.

A single chain antibody of the invention may be conjugated to a second prophylactic, therapeutic, detectable or binding moiety. The term “conjugated” is used according to its plain and ordinary meaning to indicate “to join together.” Conjugation includes the joining together of two or more compounds covalently or physically. A prophylactic or therapeutic moiety may include a nucleoside analog, a detergent, an anti-microbial (e.g., nonoxynol-9) or other molecule known in the art that that attenuates or treats HSV infection. Detectable moieties may be operatively coupled to a single chain antibody of the invention. Detectable moieties may be used in diagnostic methods for detecting exposure to or localization of HSV. A binding moiety may be operatively coupled to a single chain antibody of the invention to allow specific localization or purification of a single chain antibody. For example, a binding moiety may be operatively coupled to a single chain antibody so that the single chain antibody remains associated with a topical formulation used to administer it.

In various embodiments, an isolated polynucleotide can comprise a nucleic acid sequence encoding a single chain antibody having a microbe binding activity, a microbe binding specificity or a microbe inhibitory activity. A microbe may be a virus, bacteria or fungi that causes or may cause a sexually transmitted disease. In particular embodiments, an isolated polynucleotide can comprise a nucleic acid sequence encoding a single chain antibody having a HSV glycoprotein binding activity, a HSV glycoprotein binding specificity or a HSV inhibitory activity. The HSV glycoprotein can be a HSV glycoprotein B, C, D, E, G, H, and/or I protein (HSV gB, gC, gD, gE, gG, gH and gI, see GenBank accession numbers X14112 and NC_{—}001798 for exemplary nucleic acid and amino acid sequences, which are hereby incorporated by reference). The nucleic acid sequence may be comprised in an expression cassette and/or an expression construct. In particular embodiments a nucleic acid encoding a single chain antibody may include the nucleic acid sequence set forth in SEQ ID NO:1. A nucleic acid of the invention may be further comprised in a recombinant host cell. Polypeptides of the invention may be purified or isolated from a recombinant host cell. The recombinant host cell may include an episomally or genomically maintained expression cassette. The host may be a cell from a human, bacterium, or fungus, including yeast.

A composition can include one or more single chain antibodies having a binding activity, binding specificity or an inhibitory activity towards a sexually transmitted or pathogenic microbe. In certain embodiments, one or more single chain antibodies can have a HSV glycoprotein binding activity, a HSV glycoprotein binding specificity or a HSV inhibitory activity. Other microbes include HIV, Chlamydia, HepB and the like. The composition may further include at least a second, third, fourth, fifth, sixth, seventh, eighth, ninth, tenth or more single chain antibody with a binding affinity for the same or different sexually transmitted microbe. The second, third, fourth, fifth, sixth, seventh, eighth, ninth, tenth or more single chain antibody may bind and reduce the infectivity of one or more microbe. A microbe can be HIV, HSV, chlamydia, HepB or other sexually transmitted virus, bacterium or fungus. The composition can be comprised in a pharmaceutically acceptable composition. The pharmaceutically acceptable composition can be a topical composition. A topical composition may be a foam, a gel, suppository, or other acceptable formulation. The composition may further comprise an antiviral therapeutic agent, such as a nucleoside analog. The composition may be a contraceptive or is part of a contraceptive device in some embodiments of the invention, though it need not be. In particular embodiments, a single chain antibody may include a single chain antibody as set forth in SEQ ID NO:2. Compositions of the invention may include a second antibody. The second antibody may be a monoclonal antibody, an antibody fragment, a Fab fragment, a single chain antibody, or a bispecific antibody. Antibodies of the invention may be humanized antibodies. A humanized antibody, in which the CDRs of the antibody are derived from an antibody of a non-human animal and the framework regions and constant region are from a human antibody, may be produced, by the methods described in U.S. Pat. No. 5,225,539.

A proteinaceous composition of the invention can further include at least a second, third, fourth, fifth, sixth, seventh, eighth, ninth, tenth or more single chain antibody having a binding activity, binding specificity or inhibitory activity for at least a second, third, fourth, fifth or more sexually transmitted microbe. A second, third, fourth, fifth, or more microbe can be HSV, HIV, chlamydia, HepB, or other virus, bacterium, or fungus that causes or may cause a sexually transmitted disease.

Various embodiments of the invention include a recombinant host cell comprising an expression cassette encoding a single chain antibody having a binding activity, a binding specificity or a inhibitory activity towards a sexually transmitted microbe. In certain embodiments, an expression cassette encodes a single chain antibody having a HSV glycoprotein binding activity, a HSV glycoprotein binding specificity or a HSV inhibitory activity is contemplated. The expression cassette can be episomal or integrated into the genome of the cell.

In particular embodiments, a recombinant host cell is a bacterial cell. In other embodiments the recombinant host cell may by a mammalian, animal or human cell. The expression cassette may include a promoter that is active in the particular host cell. In certain embodiments, the promoter may be a viral promoter, such as an HSV promoter.

Embodiments of the invention include methods of producing a single chain antibody comprising: a) introducing into a cell an expression cassette encoding a single chain antibody having a binding activity, a binding specificity or an inhibitory activity towards a sexually transmitted microbe; and b) isolating the single chain antibody expressed by the cell. The binding activity or binding specificity will typically be toward a molecule (e.g., a protein) present on the surface of the target microbe, such as a carbohydrate, lipid, protein or combination thereof For example, a glycoprotein, a receptor, and/or a ligand for a cellular receptor are contemplated as targets. In particular embodiments, a single chain antibody has various characteristics such as those described herein. Methods may include the purification of the single chain antibody, which may include one or more affinity purification steps.

In certain embodiments, a method of prophylactically or therapeutically treating a subject against a sexually transmitted microbe, e.g., HSV, includes administering to a subject that is or may be exposed to the sexually transmitted microbe a proteinaceous composition comprising at least a first single chain antibody having a binding activity, binding specificity or inhibitory activity toward the sexually transmitted microbe as described herein. In particular embodiments a single chain antibody can have a HSV glycoprotein binding activity, a HSV glycoprotein binding specificity, and/or a HSV inhibitory activity. The HSV glycoprotein can be HSV gD and/ or gB. A single chain antibody of the invention binds an epitope in a molecule present on the surface of a sexually transmitted microbe. The microbe may cause or causes a sexually transmitted disease. The molecule may be a carbohydrate, a lipid, a protein or a combination thereof. In particular embodiments, one or more single chain antibodies bind an epitope on a HSV glycoprotein. In certain embodiments the HSV glycoprotein is HSV gD. The method may further include determining if the subject was exposed to HSV. The binding of a glycoprotein may in certain embodiments block the interaction of the microbe with a receptor or entry mediator present on a target cell present in organism to be infected, e.g., a human or other animal. The method may include the topical or other administration of a composition on, in or around areas of the body that may come in contact with fluid, cells, or tissue that are infected, contaminated or have associated therewith a pathogenic microbe. The composition may also be incorporated in, applied to or coated on a barrier or other protective device that is used for contraception or protection from infection with a sexually transmitted disease.

Other methods of the invention may include steps concerning determining or identifying that a subject has been exposed to a sexually transmitted microbe or determining that a subject is a risk for an infection by a sexually transmitted microbe. Thus, steps for assaying for infection or for taking a patient history are included in embodiments of the invention.

Embodiments of the invention may also include methods of attenuating the infectivity and/or the cellular entry of HSV or other sexually transmitted microbe by contacting the microbe with a single chain antibody having a microbe binding activity, a microbe binding specificity or a microbe inhibitory activity. In particular embodiments, the method involves contacting HSV with a single chain antibody composition that has a HSV glycoprotein binding activity, a HSV glycoprotein binding specificity, and/or a HSV inhibitory activity. In certain embodiments a HSV glycoprotein is the HSV gD.

In still further embodiments, methods include the assessment of single chain antibody inhibitors of HSV by preparing a first binding mixture comprising a single chain antibody and HSV and measuring the infectivity of HSV in the mixture. The infectivity of HSV or other virus may be measured by using a plaque assay. The presence of HSV in a sample may be assessed by exposing a sample to a single chain antibody that specifically binds an HSV glycoprotein, preferably HSV gD or HSV gB.

The term “binding activity” refers to the binding of an antibody to a protein or molecule of interest at a detectable level, but does not limit the binding to any one protein or molecule and binding to two or more proteins or molecules may be detected. Accordingly, “microbe binding activity” refers to binding activity with respect to a microbe.

The term “binding specifically” or “binding specificity” means binding with high avidity and/or high affinity binding, with negligible binding to other proteins, to a specific polypeptide, molecule or epitope of a protein or molecule on a microbe, which in one embodiment refers to HSV. scFv binding to its epitope on this specific molecule is preferably stronger than binding of the same scFv to any other epitope, particularly those which may be present in molecules in association with, or in the same sample or organism, as the specific polypeptide of interest. scFvs that bind specifically to a polypeptide or molecule of interest may be capable of binding other polypeptides or molecules at a weak, yet detectable, level (e.g., 10% or less of the binding shown to the polypeptide or molecule of interest). Such weak binding, or background binding, is readily discernible from the specific scFv binding to the polypeptide or molecule of interest by the use of appropriate controls, for example.

The term “inhibitory activity” refers to the functional consequence of the binding of a single chain antibody to a microbe wherein the life cycle of the microbe is disrupted. For example the infectivity and/or entry of the microbe may be attenuated, reduced, inhibited, or blocked by the binding of a single chain antibody to the microbe. One or more single chain antibodies may work in concert on the same or different surface protein and/or molecule to affect the inhibitory activity. Different single chain antibodies may attenuate interactions with the different cell types or tissue targets of the microbe.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

It is specifically contemplated that any limitation discussed with respect to one embodiment of the invention may apply to any other embodiment of the invention. Thus, any embodiment discussed with respect to HSV may be implemented with respect to other microbes. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device or method being employed to determine the value.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B. FIG. 1A: Hypothetical model illustrating the antigenic structure of gD and demonstrating the clustering of antigenic sites into seven groups, as defined by locations of amino acids bound by various monoclonal antibodies. Disulphide bonds location defined by braces. Diagram adapted from Nicola et al., 1998. Of particular relevance to this study are the locations of sites VII (amino acid residues 11-19), which is bound by antibody 1D3, and site Ib, a discontinuous epitope that includes residues 222 to 252 that is bound by antibody DL11. FIG. 1B diagrams the interface between N-terminal amino acids of gD and HveA and the approximate residues bound by monoclonal antibody 1D3 and, by inference, 1D3 scFv (adapted from Connolly et al, 2003).

FIGS. 2A-2D FIG. 2A and FIG. 2B show degenerate PCR primers used for amplification of V_{L} and V_{H} and assembly of scFv. FIG. 2C and FIG. 2D show assembly of scFv cassette using a (Gly₄Ser)₃ hinge. Alternative glycine codons were used in the overlapping region of the hinge.

FIGS. 3A-3B is a 3-D model showing the predicted structure of DL11 single chain antibody. FIG. 3A is a strand view by group, demonstrating the orientation of the kappa (top) and gamma (bottom) chains in a single plain, highlighting the residues of the (Gly₄Ser)₃ hinge attachment. FIG. 3B is a wireframe image illustrating hinge attachment sites on one side of the molecule (linked by dashed line) and Kabat CDRs clustered as marked. complementary determining regions, which form the antigen binding site, suggests correct conformation of the molecule.

FIG. 4 is a western blot demonstrating expression of DL11 scFv by E, Coli, BL21 cells transfected with p-TOPO10 containing the scFv cassette. Bacterial lysates were purified using a nickel chelation column and the reaction with anti-V5 of total lysates and various fractions from the column are shown. Lane 1, unpurified total bacterial lysate; Lane 2, nickel column flow through; Lanes 3 and 4, saline washes; Lanes 5 and 6, eluate from Ni beads; Lane 7, bacterial supernatant; Lane 8, scFv remaining on nickel column after elution; Lane 9: supernatant from uninduced bacteria.

FIG. 5 is an ELISA showing binding to plastic bound gD of bacterially expressed DL11 single chain antibody. Results are presented as a binding ratio compared with an irrelevant scFv at the same protein concentration.

FIG. 6 shows a reduction of plaque numbers in Vero cells by pre-incubation of approximately 120 PFU HSV, strain G with single chain antibodies generated from hybridomas D11 (▪), 1D3 (▴), DL2(♦) and an irrelevant CEA-specific construct (Y).

FIGS. 7A-7B shows a reduction in plaque size in the presence of DL11 scFv. Mean plaque size in absence of scFv (FIG. 7A) was 1.9±0.4 mm compared with 0.95±0.3 mm in presence of 100 mg/ml DL11 scFv (FIG. 7B). Figures represent mean of 100 plaques±standard deviation.

FIGS. 8A-8B shows the effect of DL11 scFv on HSV-1 genital disease in guinea pigs. FIG. 8A shows blisters of genital herpes 5 days after instillation of HSV-1 into vaginal vault; FIG. 8B shows a complete protection against HSV-1 by prior instillation of DL11 scFv before challenge with HSV.

FIG. 9 shows elements of T-body construction.

FIG. 10 shows structures of chimeric T-cell receptors. Heavy line: position of the immunoglobulin spacer (Ig) and transmembrane (tmCD28) sequences in the construct. Alternative signaling domains were made and comprised human Ig FcR ITAM in place of CD3 zeta and also Syk. EGFP driven by the same promoter allowed chTCR expression and T-body location to be monitored.

FIG. 11 shows the generation of T-bodies. Retroviral transduction using high titer virus (10⁶ PFU/ml) and three rounds of centrifugation (500 g) of virus and cells in Retronectin coated plates.

FIG. 12 diagrams an example of testing chimeric receptors (DL11-CD28-CD3ζ) for signaling using Jurkat cells and γ-IFN as measure of positive response.

### DESCRIPTION OF ILLUSTRATIVE EMBODIMENTS

The present invention has been made in the light of the conventional problems mentioned above. Certain embodiments of the invention provide methods and composition that include light chain variable regions and/or heavy chain variable regions of antibodies that may be engineered to provide a microbial binding activity, microbe specific binding activity and/or a microbe inhibitory activity. In particular embodiments a microbe may be HSV, however other microbial targets, such as human immunodeficiency virus (HIV, hepatitis B virus (HepB), chlamydia and other bacteria, viruses, or fungi that are known to cause sexually transmitted diseases are contemplated. In certain embodiments, polypeptides or single-chain antibodies against Herpes Simplex Virus glycoprotein D (HSVGD or gD), polynucleotides encoding such polypeptides, and therapeutic agents and methods for therapeutic or prophylactic treatment of Herpes Simplex Virus, as well as other infections are contemplated. More particularly, embodiments of the invention provide polypeptides, e.g., single-chain antibodies, characterized in that they suppress infectivity of HSV by binding to HSV glycoprotein D, polynucleotides encoding the single-chain antibodies, vectors containing comprising the polynucleotide of the invention, transformants transformed with the vectors of the invention, a process for producing the single-chain antibodies of the invention, and therapeutic agents for HSV using such single-chain antibodies or genes thereof.

The predicted amino acid sequences and 3-D structures of antibody variable regions generated from a panel of anti-gD hybridomas are described herein. The data collected is for a large number of scFvs to different mapped epitopes on a single protein and provides a basis for rapidly distinguishing between antibodies to different epitopes without resorting to labor intensive conventional epitope mapping. scFvs that recognize overlapping linear gD epitopes could be distinguished readily and rapidly by the predicted amino acid sequences both of kappa and especially gamma immunoglobulin chains. Bacterial and mammalian expression systems for generating scFv proteins may be used and binding to gD can investigated by ELISA or other methods well known to one or ordinary skill in the art. Structural modeling is one step that may be used in the identification of minimal antibody complementary determining regions, a process used when humanizing murine scFv for use in constructs for administration systemically to humans. Exemplary predicted 3-D structures of several anti-gD scFvs are presented herein.

Rapid cloning of scFv has several applications. For example, rapid cloning is a method of distinguishing between hybridomas, e.g. during monoclonal antibody production, saving considerable time and effort. Second, structural modeling readily allows identification of minimal sequences of complementary determining regions (that dictate antigen specificity), an essential step inhumanization of mouse reagents by CDR grafting. Third, certain gD scFv may be used as topical microbicides for protection against HSV. In certain embodiments, combinations with other scFvs is contemplated to produce a multivalent microbicide. Also, bacterial expression, followed by in vitro formation of scFv intra-chain disulphide bonds, can be a source of large quantities of compositions described herein.

I. Herpes Simplex Virus

Herpes simplex virus type 1 (HSV-1) and its closely related herpes simplex virus type 2 (HSV-2), cause various benign diseases, such as the common cold sore found near the lips and also genital herpes. Herpes simplex virus can also cause serious disease upon infection of the eye (e.g., keratoconjunctivitis, with the potential to lead to blindness), the brain (e.g., encephalitis). Individuals who are immunocompromized, such as a newborn baby, AIDS patient, or transplant patient, are especially vulnerable. HSV infections of immunocompromised individuals and neonates can lead to disseminated and life-threatening disease. Unlike many viruses, once an individual is infected with HSV, the virus remains latent in neurons and can be reactivated by stress or immunosuppression and cause recurrent disease. The invention, when administered to HSV-infected mothers before birth, is expected to be particularly useful for protecting the unborn child at delivery against the devastating effects of neonatal herpes.

HSV-1 contains a double-stranded linear DNA genome, 153 kilobases in length, that has been completely sequenced by McGeoch. et al., (1988); McGeoch et al., (1986); McGeoch et al. (1985); Perry and McGeoch (1988). DNA replication and virion assembly occurs in the nucleus of infected cells. Late in infection, concatemeric viral DNA is cleaved into genomic length molecules that are packaged into virions. In the CNS, herpes simplex virus spreads transneuronally followed by intraaxonal transport to the nucleus, either retrograde or anterograde, where replication occurs.

HSV virions contain over 30 proteins (virion polypeptides, VPs) including more than eight glycoproteins (including gB, gC, gD, gE, gG, gH and gI) some of which are components of the envelope spikes. The tegument contains at least two proteins of known function: αTIF (alpha trans-inducing factor, also known as VP16 and vmw65) and VHS (virion host shut off).

Five of eight viral glycoproteins are dispensable for virus growth in culture (gC, gE, gG, gI, gJ). Three glycoproteins (gB, gD, and gH) are essential and represent the minimal set of surface proteins necessary to sustain and carry out the dominant flow of events. Heparin sulfate proteoglycans appear to be the receptor molecules which are recognized by either gB or gC and which permit initial attachment of the virus. gB and gD are essential for virus penetration. Penetration occurs by direct fusion of the viral envelope with the cell membrane. Virions which attach to the plasma membrane which cannot fuse are internalized and degraded in endocytotic vesicles. Capsids are transported by the cellular cytoskeleton to nuclear pores and viral DNA is released into the nucleus where it accumulates. Single chain antibodies of the invention may target one or more of these encoded polypeptides.

Interaction of glycoprotein(s) with cellular receptors results in fusion of the envelope with the cell membrane. Endocytosis is not absolutely required, but may occur. During attachment, glycoprotein C (gC) interacts with heparan sulphate (HS), located on the cell membrane surface. This interaction is labile until other glycoproteins such as B and D (gB and gD) begin to participate in the entry process. gB also harbors a site for interaction with other glycosoaminoglycans, while gD provides a stabile attachment to cellular receptors such as the herpesvirus entry mediators (HVEM; HveA and nectin-1). Late adsorption is associated with a conformation change of gD occurring after the receptor binding.

Assessing or determining if a patient or subject is at risk of HSV infection entails the assessment of various risk factors. Risk factors include multiple sexual partners, increasing age, female gender, low socioeconomic status and human immunodeficiency virus (HIV) infection. Also, a fetus is at risk of infection during birth if the mother is infected by HSV.

For a detailed description of other infectious diseases and the various microbes that cause such disease see Mandell, Douglas and Bennett's Principles and Practice of Infectious Diseases—5^{TH} edition, Churchill Livingstone, Inc., September 1998; Sexually Transmitted Diseases, Vol. 5 Gerald L. Mandell (Editor), Michael F. Rein (Editor), Churchill Livingstone, Inc., January 1996; Sexually Transmitted Diseases in Obstetrics and Gynecology, Sebastian Faro, Lippincott Williams & Wilkins, June 2001; or Sexually Transmitted Diseases, King K. Holmes, Per-Anders Mardh (Editor), Judith Wasserheit, McGraw-Hill, January 1999; each of which is incorporated herein by reference.

II. Single Chain Antibodies (scFv)

Naturally occurring antibodies are produced by B cells and consist of four polypeptide chains held together by disulphide bonds. The polypeptides include two heavy chains composed of four immunoglobulin (Ig) domains and two light chains made up of two immunoglobulin domains. The bulk of the antibody complex is made up of constant immunoglobulin domains. These have a conserved amino acid sequence, and exhibit low variability. Different classes of constant regions in the stem of the antibody generate different isotypes of antibody with differing properties. The recognition properties of the antibody are carried by the variable regions (V_{H} and V_{L}) at the ends of the arms. Each variable domain contains three hypervariable regions known as complementarity determining regions, or CDRs. The CDRs come together in the final tertiary structure to form an antigen binding pocket.

A major advance in antibody technology was the generation of monoclonal antibodies, i.e., pure populations of antibodies with the same affinity. This was achieved by fusing B cells taken from immunized animals with myeloma cells. This generates a population of immortal hybridomas, from which the required clones can be selected. Monoclonal antibodies are very important research tools, and have been used in some therapies. However, they are very expensive and difficult to produce, and if used in a therapeutic context, can elicit and immune response which will destroy the antibody. This can be reduced in part by humanizing the antibody by grafting the CDRs from the parent monoclonal into the backbone of a human IgG antibody.

A single chain antibody is a single polypeptide which can retain the antigen binding properties of a monoclonal antibody. The variable regions from the heavy and/or light chains (V_{H} and V_{L}) are both approximately 110 amino acids long. They can be linked by a 15 amino acid linker with, for example (gly₄ser)₃, which has sufficient flexibility to allow the two domains to assemble a functional antigen binding pocket. Addition of various signal sequences allows the scFv to be targeted to different organelles within the cell, or to be secreted. Addition of the light chain constant region (Ck) allows dimerization via disulphide bonds, giving increased stability and avidity.

The variable regions for constructing the scFv can be obtained by using RT-PCR to clone out the variable regions from mRNA extracted from a hybridoma. Degenerate primers targeted to the relatively invariant regions can be used.

A description of the theory and production of single-chain antigen-binding proteins is found in Ladner et al., U.S. Pat. Nos. 4,946,778, 5,260,203, 5,455,030 and 5,518,889. The single-chain antigen-binding proteins produced under the process recited in the above U.S. Patents have binding specificity and affinity substantially similar to that of the corresponding Fab fragment. A computer-assisted method for linker design is described more particularly in Ladner et al., U.S. Pat. Nos. 4,704,692 and 4,881,175, and PCT application WO 94/12520, each of which is incorporated herein in its entirety by reference. However, the in vivo properties of sFv polypeptides are different from MAbs and antibody fragments, such Fabs. Due to their small size, sFv polypeptides clear more rapidly from the blood and penetrate more rapidly into tissues Milenic et al., 1991; Colcher et al., 1990; Yokota et al., 1992). Due to lack of constant regions, sFv polypeptides are not retained in tissues such as the liver and kidneys. Due to the rapid clearance and lack of constant regions, sFv polypeptides will have low immunogenicity. Thus, sFv polypeptides have applications in diagnosis and therapy, where rapid tissue penetration and clearance, and ease of microbial production are advantageous.

In particular embodiments of the invention a scFv can be selected for binding to a particular epitope of HSV glycoprotein D. An epitope, as used herein is a portion of a molecule that is specifically recognized by an immunoglobulin product. It is also referred to as the determinant or antigenic determinant. The epitope may be contain with the amino acid sequence of SEQ ID NO:2.

General methods for generating scFvs may be found in U.S. Pat. Nos. 5,840,300, 5,667,988, 5,658,727, 5,258,498, and 4,946,778, each of which is incorporated herein by reference. In certain embodiments, a scFv of the invention may be incorporated into a bi-specific binding agent that binds two epitopes present on one or more pathogens. Further description of single chain antibodies, single domain antibodies, and bispecific binding agents can be found, for example, in Malecki et al. (2002); Conrath et al. (2001); Desmyter, et al. (2001); Kostelney, et al. (1992); U.S. Pat. Nos. 5,932,448; 5,532,210; 6,129,914; 6,133,426, each of which is incorporated herein in its entirety by reference.

Different parts of the antibodies can be joined by means of conventional methods or constructed as a contiguous protein by means of recombinant DNA techniques, e.g., in such a way that a nucleic acid molecule coding for a chimeric or humanized antibody chain is expressed in order to construct a contiguous protein (e.g., see Mack (1995)).

In one aspect, a single-chain antibody with the following Fv fragments is used: sc-Fv fragment of a monoclonal antibody against a first HSV glycoprotein (e.g., gD) and an sc-Fv fragment of a monoclonal antibody against a second HSV glycoprotein (e.g., gB) to form a bispecific antibody. Compared to conventional bispecific antibodies, bispecific single-chain antibodies have the advantage that they consist of only one protein chain and thus their composition is exactly defined. They have a low molecular weight of normally <60 kD and can be produced easily and on a large scale in suitable cell lines, e.g., in CHO cells, using recombinant techniques. One advantage, however, is that they have no constant antibody domains and thus only activate T-lymphocytes to lysis when these are bound to their target cells. Therefore, single-chain antibodies are often superior to conventional bispecific antibodies as their clinical use entails fewer or less severe side effects. Also, antibody fragments can be produced on a relatively large scale in prokaryotic cells, thus facilitating their production. Furthermore, the relatively small size of single-chain antibody fragments makes them less likely than whole antibodies to provoke an immune response in a recipient.

A wide variety of expression systems are available in the art for the production of scFva, as well as other antibody fragments. For example, suitable to the large-scale production of antibody fragments and antibody fusion proteins are expression systems of both prokaryotic and eukaryotic origin. Particularly advantageous are expression systems that permit the secretion of large amounts of antibody fragments into the culture medium.

Eukaryotic expression systems for large-scale production of antibody fragments and antibody fusion proteins have been described that are based on mammalian cells, insect cells, plants, transgenic animals, and lower eukaryotes. For example, the cost-effective, large-scale production of antibody fragments can be achieved in yeast fermentation systems. Large-scale fermentation of these organisms is well known in the art and is currently used for bulk production of several recombinant proteins. Yeasts and filamentous fungi are accessible for genetic modifications and the protein of interest may be secreted into the culture medium. In addition, some of the products comply with the GRAS (Generally Regarded as Safe) status-they do not harbor pyrogens, toxins, or viral inclusions.

The methylotrophic and other yeasts like Candida boidinii, Hansenula polymorpha, Pichia methanolica, and Pichia pastoris are well know systems for the production of heterologous proteins. High levels of proteins in milligram to gram quantities can be obtained and scaling up to fermentation for industrial applications is possible.

The P. pastoris system is used in several industrial-scale production processes. For example, the use of Pichia for the expression of scFv fragments as well as recombinant antibodies and fragments thereof have been described (Ridder et al., 1995; Anadrade et al., 2000; Pennell et al., 1998). In shake-flask cultures, levels of 250 mg/L to over 1 g/L of scFv or VHH can be achieved (Eldin et al., 1997); Freyre et al., 2000).

Similar expression systems for scFv have been described for Saccharomyces cerevisiae, Schizosaccharomyces pombe, Yarrowia lipolytica, and Kluyveromyces lactis (Horwitz et al., 1988; Davis et al., 1991; Swennen et al., 2002). Filamentous fungi, such as Trichoderma and Aspergillus, have the capacity to secrete large amounts of proteins. This property may be exploited for the expression of scFvs (Radzio et al., 1997; Punt et al., 2002; Verdoes et al., 1995; Gouka et al., 1997; Ward et al., 1990; Archer et al., 1994); Durand et al., 1988; Keranen et al., 1995; Nevalainen et al., 1994; Nyyssonen et al., 1993; and Nyyssonen et al., PCT WO 92/01797 1992).

A. Antibody Conjugates

Further aspects of the invention include antibody conjugates comprising a HSV gD single chain antibody linked to another agent such as, but not limited to, a therapeutic agent, a detectable label, a cytotoxic agent, a chemical, a toxic, an enzyme inhibitor, a pharmaceutical agent, etc. Diagnostic antibody conjugates may be used both in in vitro diagnostics, as in a variety of immunoassays, and in in vivo diagnostics, such as in imaging technology.

Certain antibody conjugates include those intended primarily for use in vitro, where the antibody is linked to a secondary binding ligand or to an enzyme (an enzyme tag) that will generate a colored product upon contact with a chromogenic substrate. Examples of suitable enzymes include urease, alkaline phosphatase, (horseradish) peroxidase and glucose oxidase. Preferred secondary binding ligands are biotin and avidin or streptavidin compounds. The use of such labels is well known to those of skill in the art and is described, for example, in U.S. Pat. Nos. 3,817,837; 3,850,752; 3,939,350; 3,996,345; 4,277,437; 4,275,149 and 4,366,241; each incorporated herein by reference. Other antibody conjugates, intended for functional utility, include those where the antibody is conjugated to an antiviral compound such as nucleoside analogs.

B. Radiolabeled Antibody Conjugates

In using an antibody-based molecule as an in vivo diagnostic agent to provide an image of brain and neurons, for example, magnetic resonance imaging, X-ray imaging, computerized emission tomography and other imaging technologies may be employed. In the antibody-imaging constructs of the invention, the antibody portion used will generally bind to HSV, e.g., binding a HSV gD antigen or epitope, and the imaging agent will be an agent detectable upon imaging, such as a paramagnetic, radioactive or fluorescent agent.

Many appropriate imaging agents are known in the art, as are methods for their attachment to antibodies (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the antibody (U.S. Pat. No. 4,472,509). scFvs also may be reacted with an enzyme in the presence of a coupling agent such as glutaraldehyde or periodate. Conjugates with fluorescein markers are prepared in the presence of these coupling agents or by reaction with an isothiocyanate.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred.

Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and yttrium⁹⁰. ¹²⁵I is often being preferred for use in certain embodiments, and technicium^{99m} and indium¹¹¹ are also often preferred due to their low energy and suitability for long range detection.

Radioactively labeled binding agents or antibodies of the present invention may be produced according to well-known methods in the art.

III. Proteinaceous Compositions

In certain embodiments, the present invention concerns compositions comprising at least one proteinaceous molecule. The proteinaceous molecule may be a modulator of HSV life cycle through binding of a HSV glycoprotein, in particular glycoprotein D, or it may be used as a candidate substance to be screened as a modulator of HSV glycoprotein activity. The proteinaceous molecule may also be used, for example, in a pharmaceutical composition for the delivery of a therapeutic agent or as part of a screening assay to identify HSV modulators. As used herein, a “proteinaceous molecule,” “proteinaceous composition,” “proteinaceous compound,” “proteinaceous chain” or “proteinaceous material” generally refers, but is not limited to, a protein of greater than about 100 amino acids or the full length endogenous or engineered sequence translated from a gene; a polypeptide of greater than about 100 amino acids; and/or a peptide of from about 3 to about 100 amino acids. All the “proteinaceous” terms described above may be used interchangeably herein. In particular, a light chain variable region, heavy chain variable region, and/or a single chain antibody may be referred to as a “proteinaceous molecule,” “proteinaceous composition,” “proteinaceous compound,” “proteinaceous chain” or “proteinaceous material.”

In certain embodiments, the size of the at least one proteinaceous molecule may comprise, but is not limited to 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 or greater amino molecule residues, and any range derivable therein. Furthermore, such proteinaceous molecules may include 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, or 306 contiguous amino acid residues from SEQ ID NO:2, or variants thereof.

In certain embodiments, a single chain antibody comprises a sequence in which about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100% of the amino acid sequence is identical to the amino acid sequence of SEQ ID NO:2 or similar sequences as identified by the methods described herein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid, including but not limited to those shown on Table 1 below.

TABLE 1
Modified and Unusual Amino Acids

| Abbr. | Amino Acid |
|---|---|
| Aad | 2-Aminoadipic acid |
| Baad | 3-Aminoadipic acid |
| Bala | β-alanine, β-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| Ahyl | allo-Hydroxylysine |
| 3Hyp | 3-Hydroxyproline |
| 4Hyp | 4-Hydroxyproline |
| Ide | Isodesmosine |
| Aile | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials.

In certain embodiments a proteinaceous compound may be purified. Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that it will allow the composition to be more precisely or easily applied to the tissue and to be maintained in contact with the tissue. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred. In certain embodiments the proteinaceous composition may be comprised in a gel or foam.

A. Isolating Proteinaceous Compounds

A polypeptide that binds, specifically binds, or inhibits a microbe, e.g., HSV may be obtained according to various standard methodologies that are known to those of skill in the art. For example, antibodies or other binding agents specific for HSV glycoproteins may be used in inimunoaffinity protocols to isolate the respective polypeptide from infected cells, in particular, from infected cell lysates. Antibodies are advantageously bound to supports, such as columns or beads, and the immobilized antibodies can be used to pull the polypeptides of interest out of the cell lysate. These antibodies or binding agents may recognize generally HSV glycoproteins, specifically HSV glycoprotein D, and/or a generally or specifically recognize a peptide or polypeptide that is fused or conjugated, covalently or non-covalently, to the polypeptide of interest.

In other embodiments, HSV polypeptides may be used to screen for binding agents such as monoclonal antibodies or scFv antibodies.

Alternatively, expression vectors may be used to generate the polypeptide of interest. A wide variety of expression vectors may be used, including viral expression vectors. The structure and use of these vectors is discussed further, below. Such vectors may significantly increase the amount of a polypeptide of interest in the cells, and may permit less selective purification methods such as size fractionation (chromatography, centrifugation), ion exchange or affinity chromatography, and even gel purification. Alternatively, the expression vector may be provided directly to target cells, again as discussed further, below.

Polypeptides of interest, i.e., polypeptides that bind, specifically bind, neutralize, and/or inhibit the activity or life cycle of a microbe, according to the present invention, may advantageously be fragmented in the generation of reagents such as single chain antibodies and fragments thereof. This can be accomplished by recombinant techniques to produce specific fragment or fragments of an antibody of interest. It may be that the neutralizing and microbe-inhibiting functions of an antibody of interest reside in distinct domains or regions of the protein. If such is the case, the ability to make domain or region-specific reagent(s) now has significance.

It is expected that changes may be made in the sequence of a polypeptide of interest while retaining a molecule having the structure and function of the polypeptide of interest. For example, certain amino acids may be substituted for other amino acids in a protein structure without appreciable loss of interactive capacity with structures such as, for example, substrate-binding regions or CDR. These changes are termed “conservative” in the sense that they preserve the structural and, presumably, required functional qualities of the starting molecule.

B. Variants

Conservative amino acid substitutions generally are based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as equivalent.

In making such changes, the hydropathic index of amino acids also may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It also is understood in the art that the substitution of like amino acids can be made effectively on the basis of hydrophilicity. As detailed in U.S. Pat. No. 4,554,101, incorporated herein by reference, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

Numerous scientific publications have been devoted to the prediction of secondary structure, and to the identification of epitopes, from analyses of amino acid sequences (Chou and Fasman, 1974a,b; 1978a,b; 1979). Computer programs are currently available to assist with predicting antigenic portions and epitopic core regions of proteins. Examples include those programs based upon the Jameson-Wolf analysis (Jameson and Wolf, 1988; Wolf et al., 1988), the program PepPlot® (Brutlag et al., 1990; Weinberger et al., 1985), and other new programs for protein tertiary structure prediction (Fetrow and Bryant, 1993). The methods may be used to identify other epitopes to which antibodies may be raised and engineeted as set forth herein.

Two designations for amino acids are used interchangeably throughout this application, as is conunon practice in the art. Alanine=Ala (A); Arginine=Arg (R); Aspartate=Asp (D); Asparagine=Asn (N); Cysteine=Cys (C); Glutamate=Glu (E); Glutamine=Gln (Q); Glycine=Gly (G); Histidine=His (H); Isoleucine=Ile (I); Leucine=Leu (L); Lysine=Lys (K); Methionine=Met (M); Phenylalanine=Phe (F); Proline=Pro (P); Serine=Ser (S); Threonine=Thr (T); Tryptophan=Trp (W); Tyrosine=Tyr (Y); Valine=Val (V).

IV. Nucleic Acids

In particular aspects of the invention, a nucleic acid encodes for or comprises a transcribed nucleic acid. In other aspects, for example, a nucleic acid may comprise a nucleic acid segment of SEQ ID NO:1, or a biologically functional equivalent thereof.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 8 and about 100 nucleobases in length. The term “polynucleotide” refers to at least one molecule of greater than about 100 nucleobases in length.

Herein certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

“Isolated substantially away from other coding sequences” means that the gene of interest forms the significant part of the coding region of the nucleic acid, or that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

A. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in he art, particularly when a nucleotide comprises derivatives or analogs of a naturally occurring 5-carbon sugar or phosphorus moiety.

B. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266 032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which are incorporated herein by reference.

A non-limiting example of an enzymatically produced nucleic acid include one produced by enzymes in amplification reactions such as PCR™ (see for example, U.S. Pate. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

C. Purification of Nucleic Acids

A nucleic acid may be purified on polyacrylamide gels, cesium chloride centrifugation gradients, or by any other means known to one of ordinary skill in the art (see for example, Sambrook et al., 2001, incorporated herein by reference).

In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

D. Nucleic Acid Segments

In certain embodiments, the nucleic acid is a nucleic acid segment. As used herein, the term “nucleic acid segment,” are smaller fragments of a nucleic acid, such as for non-limiting example, those that encode only part of the SEQ ID NO:1. Thus, a “nucleic acid segment” may comprise any part of a gene sequence, of from about 8 nucleotides to the full length of the SEQ ID NO:1.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:
n to n+y
where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. This algorithm would be applied to each of SEQ ID NO:1. As used herein, a “probe” generally refers to a nucleic acid used in a detection method or composition. As used herein, a “primer” generally refers to a nucleic acid used in an extension or amplification method or composition.

In a non-limiting example, one or more nucleic acid constructs may be prepared that include a contiguous stretch of nucleotides identical to or complementary to SEQ ID NO:1. A nucleic acid construct may be about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 60, about 70, about 80, about 90, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include bout 11, about 12, about 13, about 14, about 15, about 16, about 17, about 18, about 19, about, 20, about 21, about 22, about 23, about 24, about 25, about 26, about 27, about 28, about 29, about 30, about 35, about 40, about 50, about 60, about 70, about 80, about 90, about 100, about 125, about 150, about 175, about 200, about 500, about 1,000, about 10,000, about 50,000, about 100,000, about 250,00, about 500,00, about 1,000,000 or more bases.

E. Nucleic Acid Complements

The present invention also encompasses a nucleic acid that is complementary to a SEQ ID NO:1 or similar nucleic acids identified by the methods described herein. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule of SEQ ID NO:1 or similar nucleic acids identified by the methods described herein during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

F. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

G. Genetic Degeneracy

The term “functionally equivalent codon” is used herein to refer to codons that encode the same amino acid, such as the six codons for arginine and serine, and also refers to codons that encode biologically equivalent amino acids. For optimization of expression in human cells, the codons are shown in Table 3 in preference of use from left to right. Thus, the most preferred codon for alanine is thus “GCC”, and the least is “GCG” (see Table 3 below). Codon usage for various organisms and organelles can be found at the website kazusa.orjp/codon/, incorporated herein by reference, allowing one of skill in the art to optimize codon usage for expression in various organisms using the disclosures herein. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria, chloroplasts and the like, based on the preferred codon usage as would be known to those of ordinary skill in the art.

TABLE 3
Preferred Human DNA Codons

| Amino Acids | | | Codons | | | | | |
|---|---|---|---|---|---|---|---|---|
| Alanine | Ala | A | GCC | GCT | GCA | GCG | | |
| Cysteine | Cys | C | TGC | TGT | | | | |
| Aspartic acid | Asp | D | GAC | GAT | | | | |
| Glutamic acid | Glu | E | GAG | GAA | | | | |
| Phenylalanine | Phe | F | TTC | TTT | | | | |
| Glycine | Gly | G | GGC | GGG | GGA | GGT | | |
| Histidine | His | H | CAC | CAT | | | | |
| Isoleucine | Ile | I | ATC | ATT | ATA | | | |
| Lysine | Lys | K | AAG | AAA | | | | |
| Leucine | Leu | L | CTG | CTC | TTG | CTT | CTA | TTA |
| Methionine | Met | M | ATG | | | | | |
| Asparagine | Asn | N | AAC | AAT | | | | |
| Proline | Pro | P | CCC | CCT | CCA | CCG | | |
| Glutamine | Gln | Q | CAG | CAA | | | | |
| Arginine | Arg | R | CGC | AGG | CGG | AGA | CGA | CGT |
| Serine | Ser | S | AGC | TCC | TCT | AGT | TCA | TCG |
| Threonine | Thr | T | ACC | ACA | ACT | ACG | | |
| Valine | Val | V | GTG | GTC | GTT | GTA | | |
| Tryptophan | Trp | W | TGG | | | | | |
| Tyrosine | Tyr | Y | TAC | TAT | | | | |

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

Excepting intronic and flanking regions, and allowing for the degeneracy of the genetic code, nucleic acid sequences that have between about 70% and about 79%; or more preferably, between about 80% and about 89%; or even more particularly, between about 90% and about 99%; of nucleotides that are identical to the nucleotides of SEQ ID NO:1, or similar nucleic acids identified by the methods described herein, will be nucleic acid sequences that are “essentially as set forth in SEQ ID NOS:1 or similar nucleic acids identified by the methods described herein”.

H. Vectors and Expression Constructs

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Sambrook et al., 2001 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operable linked coding sequence in a particular host cell. In addition to control sequences that govern transcription and translation, vectors and expression vectors may contain nucleic acid sequences that serve other functions as well and are described infra.

In order to express a single chain antibody of the invention it is necessary to provide a single chain antibody gene in an expression vehicle. The appropriate nucleic acid can be inserted into an expression vector by standard subcloning techniques. For example, an E. coli or baculovirus expression vector is used to produce recombinant polypeptide in vitro. The manipulation of these vectors is well known in the art. In one embodiment, the protein is expressed as a fusion protein with β-gal, allowing rapid affinity purification of the protein.

Examples of such fusion protein expression systems are the glutathione S-transferase system (Pharmacia, Piscataway, N.J.), the maltose binding protein system (NEB, Beverley, Mass.), the FLAG system (IBI, New Haven, Conn.), and the 6×His system (Qiagen, Chatsworth, Calif.).

Some of these fusion systems produce recombinant protein bearing only a small number of additional amino acids, which are unlikely to affect the functional capacity of the recombinant protein. For example, both the FLAG system and the 6×His system add only short sequences, both of which are known to be poorly antigenic and which do not adversely affect folding of the protein to its native conformation. Other fusion systems produce proteins where it is desirable to excise the fusion partner from the desired protein. In another embodiment, the fusion partner is linked to the recombinant protein by a peptide sequence containing a specific recognition sequence for a protease. Examples of suitable sequences are those recognized by the Tobacco Etch Virus protease (Life Technologies, Gaithersburg, Md.) or Factor Xa (New England Biolabs, Beverley, Mass.).

Recombinant bacterial cells, for example E. coli, are grown in any of a number of suitable media, for example LB, and the expression of the recombinant polypeptide induced by adding IPTG to the media or switching incubation to a higher temperature. After culturing the bacteria for a further period of between 2 and 24 hours, the cells are collected by centrifugation and washed to remove residual media. The bacterial cells are then lysed, for example, by disruption in a cell homogenizer and centrifuged to separate the dense inclusion bodies and cell membranes from the soluble cell components. This centrifugation can be performed under conditions whereby the dense inclusion bodies are selectively enriched by incorporation of sugars such as sucrose into the buffer and centrifugation at a selective speed.

If the recombinant protein is expressed in the inclusion bodies, as is the case in many instances, these can be washed in any of several solutions to remove some of the contaminating host proteins, then solubilized in solutions containing high concentrations of urea (e.g. 8M) or chaotropic agents such as guanidine hydrochloride in the presence of reducing agents such as β-mercaptoethanol or DTT (dithiothreitol).

Under some circumstances, it may be advantageous to incubate the polypeptide for several hours under conditions suitable for the protein to undergo a refolding process into a conformation which more closely resembles that of the native protein. Such conditions generally include low protein concentrations less than 500 μg/ml, low levels of reducing agent, concentrations of urea less than 2 M and often the presence of reagents such as a mixture of reduced and oxidized glutathione which facilitate the interchange of disulphide bonds within the protein molecule.

The refolding process can be monitored, for example, by SDS-PAGE or with antibodies which are specific for the native molecule (which can be obtained from animals vaccinated with the native molecule). Following refolding, the protein can then be purified further and separated from the refolding mixture by chromatography on any of several supports including ion exchange resins, gel permeation resins or on a variety of affinity columns.

In yet another embodiment, the expression system used is one driven by the baculovirus polyhedron promoter. The gene encoding the protein can be manipulated by standard techniques in order to facilitate cloning into the baculovirus vector. A preferred baculovirus vector is the pBlueBac vector (Invitrogen, Sorrento, Calif.). The vector carrying the gene of interest is transfected into Spodoptera frugiperda (Sf9) cells by standard protocols, and the cells are cultured and processed to produce the recombinant protein. Mammalian cells exposed to baculoviruses become infected and may express the foreign gene only. This way one can transduce all cells and express the gene in dose dependent manner.

There also are a variety of eukaryotic vectors that provide a suitable vehicle in which recombinant polypeptide can be produced. HSV itself has been used in tissue culture to express a large number of exogenous genes as well as for high level expression of its endogenous genes.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a gene and translation of a RNA into a gene product. In other embodiments, expression only includes transcription of the nucleic acid, for example, to generate antisense constructs.

In preferred embodiments, the nucleic acid is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Where a bacterial cell is targeted, it is preferable to position the nucleic acid coding region under the control of an appropriate bacterial promoter. Generally speaking, such a promoter might include either a human or viral promoter. Preferred promoters include those derived from HSV or the α4 promoter. Another preferred embodiment is the tetracycline controlled promoter.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters which are well-known in the art to achieve expression of a transgene is contemplated as well, provided that the levels of expression are sufficient for a given purpose. Tables 4 and 5 list several elements/promoters which may be employed, in the context of the present invention, to regulate the expression of a transgene. This list is not exhaustive of all the possible elements involved but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional relation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression of a transgene. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 4
PROMOTER

Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Muscle Creatine Kinase
Prealbumin (Transthyretin)
Elastase I
Metallothionein
Collagenase
Albumin Gene
α-Fetoprotein
τ-Globin
β-Globin
c-fos
c-HA-ras
Insulin
Neural Cell Adhesion Molecule (NCAM)
α_{1-Antitrypsin}
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

TABLE 5

| Element | Inducer |
|---|---|
| MT II | Phorbol Ester (TPA) |
| | Heavy metals |
| MMTV (mouse mammary tumor virus) | Glucocorticoids |
| β-Interferon | poly(rI)X |
| | poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle |
| | Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone α Gene | Thyroid Hormone |

One will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements (Bittner et al., 1987).

In various embodiments of the invention, the expression construct may comprise a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986) and adeno-associated viruses. Retroviruses also are attractive gene transfer vehicles (Nicolas and Rubenstein, 1988; Temin, 1986) as are vaccinia virus (Ridgeway, 1988) and adeno-associated virus (Ridgeway, 1988). Such vectors may be used to (i) transform cell lines in vitro for the purpose of expressing proteins of interest or (ii) to transform cells in vitro or in vivo to provide therapeutic polypeptides in a gene therapy scenario.

Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

1. Viral Vectors

Viral vectors are a kind of expression construct that utilize viral sequences to introduce nucleic acid and possibly proteins into a cell. The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Vector components of the present invention may be a viral vector that encode one or more candidate substance or other components such as, for example, an immunomodulator or adjuvant for the candidate substance. Non-limiting examples of virus vectors that may be used to deliver a nucleic acid of the present invention are described below.

A particular method for delivery of the nucleic acid involves the use of an adenovirus expression vector. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

a. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the candidate substances of the present invention as it has a high frequency of integration and it can infect nondividing cells, thus making it useful for delivery of genes into mammalian cells, for example, in tissue culture (Muzyczka, 1992) or in vivo. Details concerning the generation and use of rAAV vectors are described in U.S. Pat. Nos. 5,139,941 and 4,797,368, each incorporated herein by reference.

b. Retroviral Vectors

Retroviruses have promise as a delivery vector due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid (e.g., one encoding a single chain antibody described herein) is inserted into the viral genome in the place of certain viral sequences to produce a virus that is replication-defective. In order to produce virions, a packaging cell line containing the gag, pol, and env genes but without the LTR and packaging components is constructed Mann et al., 1983).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell is described in U.S. Pat. No. 5,994,136, incorporated herein by reference.

c. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

2. Vector Delivery and Cell Transformation

Suitable methods for nucleic acid delivery for transformation of a cell, a tissue or an organism for use with the current invention are believed to include virtually any method by which a nucleic acid (e.g., DNA) can be introduced into a cell, a tissue or an organism, as described herein or as would be known to one of ordinary skill in the art. Such methods include, but are not limited to, direct delivery of DNA such as by ex vivo transfection (Wilson et al., 1989, Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (WO 94/09699 and WO 95/06128; U.S. Pat. Nos. 5,610,042, 5,322,783, 5,563,055, 5,550,318, 5,538,877 and 5,538,880, each of which is incorporated herein by reference); by agitation with silicon carbide fibers (Kaeppler et al., 1990; U.S. Pat. Nos. 5,302,523 and 5,464,765, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, cell(s), tissue(s) or organism(s) may be stably or transiently transformed.

3. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

In certain embodiments, the host cell or tissue may be comprised in at least one organism. In certain embodiments, the organism may be, but is not limited to, a prokaryote (e.g., a eubacteria, an archaea) or an eukaryote, as would be understood by one of ordinary skill in the art (see, for example, webpage phylogeny.arizona.edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org) or through various vendors and commercial sources that cell expression systems. An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5α, JM109, and KC8, bacilli such as Bacillus subtilis; and other enterobacteriaceae such as Salmonella typhimurium, Serratia marcescens, various Pseudomonas species, as well as a number of commercially available bacterial hosts such as SURE® Competent Cells and SOLOPACK™ Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

It is an aspect of the present invention that the nucleic acid compositions described herein may be used in conjunction with a host cell. For example, a host cell may be transfected using all or part of SEQ ID NO: 1 or similar sequences.

4. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBA® 2.0 from INVITROGEN® and BACPACK™ BACULOVIRUS EXPRESSION SYSTEM FROM CLONTECH®.

Other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia inethanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed,” i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g., 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

V. T-Body Immunotherapy For Herpes Simplex

In other embodiments of the invention, a single chain antibody as described herein may be fused to transmembrane and intracellular components of the T-cell receptor to used to produce a T-body. Control of HSV infection is hampered by evasion of CD8+ T cell-mediated immune responses caused by low MHC-1 expression by neurons and infected cells. T-bodies are lymphocytes whose antigenic targets have been redirected to predefined alternative targets, different to those encoded by their endogenous receptors. T-bodies are not MHC dependent, because they use chimeric T-cell receptors (chTCR) comprising an antibody scFv coupled to T-cell signaling domains. For example, a panel of anti-gD scFv was constructed and spliced to transmembrane and intracellular regions of human CD28 plus ITAMs derived from the human TCR zeta chain. In addition, a GFP cassette was added to chTCR constructs to facilitate identification and tracking of T-bodies. Host cells were efficiently transduced using a retroviral vector and shown to proliferate and secrete interferon-gamma on exposure to plastic bound gD. To demonstrate that anti-gD T-bodies recognize infected cells in an MHC-independent manner in vivo, murine T-bodies tested in a mouse model of neuronal infection. T-bodies represent a novel immunotherapeutic approach to HSV infection.

T-bodies with receptors constructed using anti-HSV gD scFv may be useful in the treatment of severe manifestations of herpes simplex or other microbes, especially drug resistant infections in immunocompromised persons.

VI. HSV Detection and Diagnosis

In general, HSV may be detected in a patient based on the presence of one or more HSV proteins and/or polynucleotides encoding such proteins in a biological sample (for example, blood, sera, sputum urine and/or other appropriate cells or tissues) obtained from a subject or patient. In other words, such proteins may be used as markers to indicate the presence or absence of HSV in a subject or patient. The binding agents provided herein, i.e., single chain antibodies, generally permit detection of the level of antigen and/or epitope that binds to the agent in the biological sample.

There are a variety of assay formats known to those of ordinary skill in the art for using a binding agent to detect polypeptide markers in a sample. See, for example, Harlow and Lane, Antibodies: A Laboratory Manual, Cold Spring Harbor Laboratory, 1988. In general, the presence or absence of HSV in a patient may be determined by contacting a biological sample obtained from a patient with a binding agent and detecting in the sample a level of polypeptide that binds to the binding agent.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length HSV proteins and portions thereof, including HSV glycoprotein D, to which a binding agent binds, as described above.

The solid support may be any material known to those of ordinary skill in the art to which the protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 .mu.g, and preferably about 100 ng to about 1 .mu.g, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane.

Of course, numerous other assay protocols exist that are suitable for use with the HSV proteins or binding agents of the present invention. The above descriptions are intended to be exemplary only. For example, it will be apparent to those of ordinary skill in the art that the above protocols may be readily modified to use HSV polypeptides to detect antibodies that bind to such polypeptides in a biological sample. The detection of such protein-specific antibodies can allow for the identification of HSV infection.

As noted above, to improve sensitivity, multiple HSV protein markers may be assayed within a given sample. It will be apparent that binding agents specific for different HSV polypeptides may be combined within a single assay. Further, multiple primers or probes may be used concurrently. The selection of HSV protein markers may be based on routine experiments to determine combinations that results in optimal sensitivity. In addition, or alternatively, assays for HSV proteins provided herein may be combined with assays for other known HSV antigens.

The present invention further provides kits for use within any of the above diagnostic and/or therapeutic methods. Such kits typically comprise two or more components necessary for performing a diagnostic and/or therapeutic assay and will further comprise instructions for the use of said kit. Components may be compounds, reagents, containers and/or equipment. For example, one container within a diagnostic kit may contain a monoclonal antibody or fragment thereof that specifically binds to a HSV protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

VII. Pharmaceutical Formulation

In various embodiments of the present invention, a method of treatment or prophylaxis for a microbial infection is contemplated. Examples of microbial infection contemplated for treatment include HIV, HSV, HepB, chlamydia, and other infectious microbes described herein and in literature referenced may be treated.

An effective amount of the pharmaceutical composition, generally, is defined as that amount sufficient to detectably and repeatedly ameliorate, reduce, minimize or limit the extent of the infection, disease or its symptoms. More rigorous definitions may apply, including elimination, eradication or cure of disease.

Pharmaceutical compositions of the present invention comprise an effective amount of one or more single chain antibody having a binding activity, a specific binding activity, and/or an inhibitory activity towards an infectious microbe, and/or an additional agent(s) dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least 1, 2, 3, 4, 5, 6, 7, 8, 9, 10 or more single chain antibody of the invention and/or additional agent(s) dissolved or dispersed in a pharmaceutically acceptable carrier as will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drags, drug stabilizers, gels, foams, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 1990, incorporated herein by reference). Except insofar as any conventional carrier is incompatible with the active ingredient, its use in the therapeutic or pharmaceutical compositions is contemplated. Carriers are suitable for application to the vaginal tract, oral cavity, gastrointenstinal tract, rectum and other mucosal surfaces. Particularly, carriers are mucoadhesive gels. Suitable carriers may comprise organic solvents, emulsifiers, gelling agents, moisturizers, stabilizers, wetting agents, time release agents, sequestering agents, dyes, perfumes and other components commonly employed in pharmaceutical compositions for administration to mucous membranes.

The single chain antibodies of the invention may be formulated into a composition in a free base, neutral or salt form. Pharmaceutically acceptable includes the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

Sterile injectable solutions may be prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

Any of the conventional methods for administration of a proteinaceous composition as described herein are applicable. These include, but are not limited to oral and /or topical application on a solid physiologically acceptable base or in a physiologically acceptable dispersion, parenterally, by injection or the like. Compositions of the invention may be administered parenterally, by injection, for example, either subcutaneously or intramuscularly. Additional formulations which are suitable for other modes of administration include, in some cases, topical and oral formulations. In other embodiments, one may use eye drops, nasal solutions or sprays, aerosols or inhalants in the present invention. Such compositions are generally designed to be compatible with the target tissue type. In a non-limiting example, vaginal, solutions are usually aqueous, foam or gel solutions designed to be administered in suppositories, on protective barriers, in drops or in sprays. Topical solutions are prepared so that they are similar in many respects to bodily secretions, so that normal physiological action is maintained. In addition, antimicrobial preservatives or appropriate drug stabilizers, if required, may be included in the formulation.

In certain preferred embodiments, a proteinaceous composition as described herein may comprise one or more binders, excipients, disintegration agents, lubricants, and combinations thereof. In certain embodiments, a composition may comprise one or more of the following: a binder, such as, for example, gum tragacanth, acacia, cornstarch, gelatin, hydrogel or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both. Formulations may contain about 10 to about 95% of active ingredient, preferably about 25 to about 70%.

In certain embodiments, a proteinaceous composition as described herein may comprise, for example, at least about 0.1% of an active compound. In other embodiments, an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more of antigen or total protein per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In many instances, it will be desirable to have multiple administrations of a proteinaceous composition as described herein.

“Unit dose” is defined as a discrete amount of an active composition dispersed in a suitable carrier. For example, in accordance with the present methods, viral doses include a particular mass of protein.

VIII. Three Dimensional Modeling of scFV

It is well known that the three-dimensional structures of similar proteins are more conservative than their primary structures. So long as 50% homology exists in the amino acid sequence, the space deviations of α-carbon atoms in the main chain would be less than 0.3 nm with a root mean square bias of 0.1 nm. Replacement of amino acid residues often happen at the turns on the surface of the protein, it has little influence on the structure of the main backbone of the protein molecule, especially the hydrophobic core (Blundell et al. Nature 326: 347-52, 1987). Thus it is feasible to predict the three-dimensional structure of a protein with reference to sterically defined proteins with sequence homology. In construction of the Sc3F3, variable regions from the heavy and light chains (V_{H}, V_{L}) of 3F3 were linked together with a peptide. The interactions between V_{H} and V_{L} only influence their relative sterical positions and the conformations of several amino acid residues intervening in the contact of the two chains, but there is little impact on the integral chain structure. Hence it is tenable to model V_{H} and V_{L} separately, then connects them by computer-aided modeling.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Materials and Methods

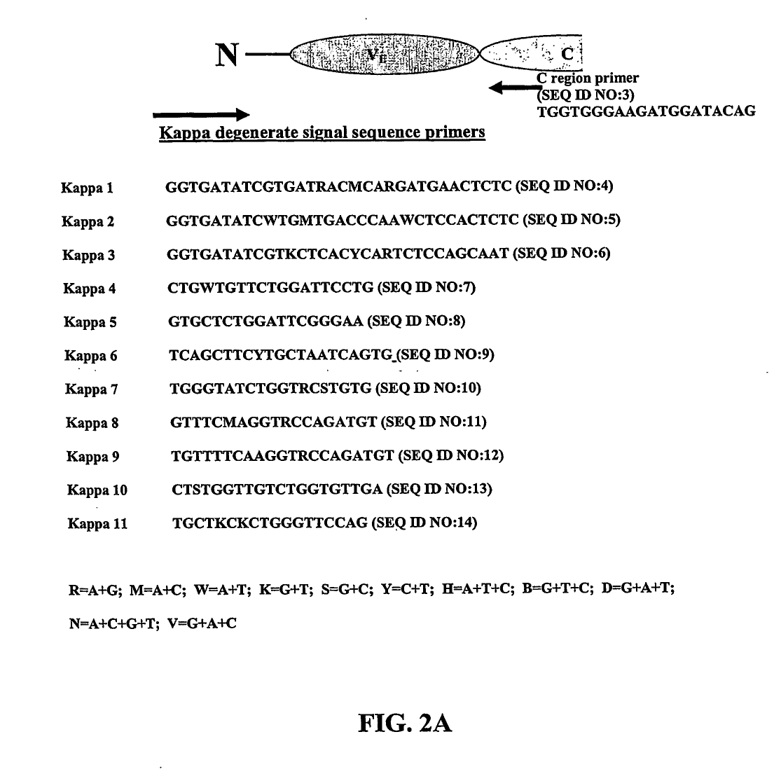

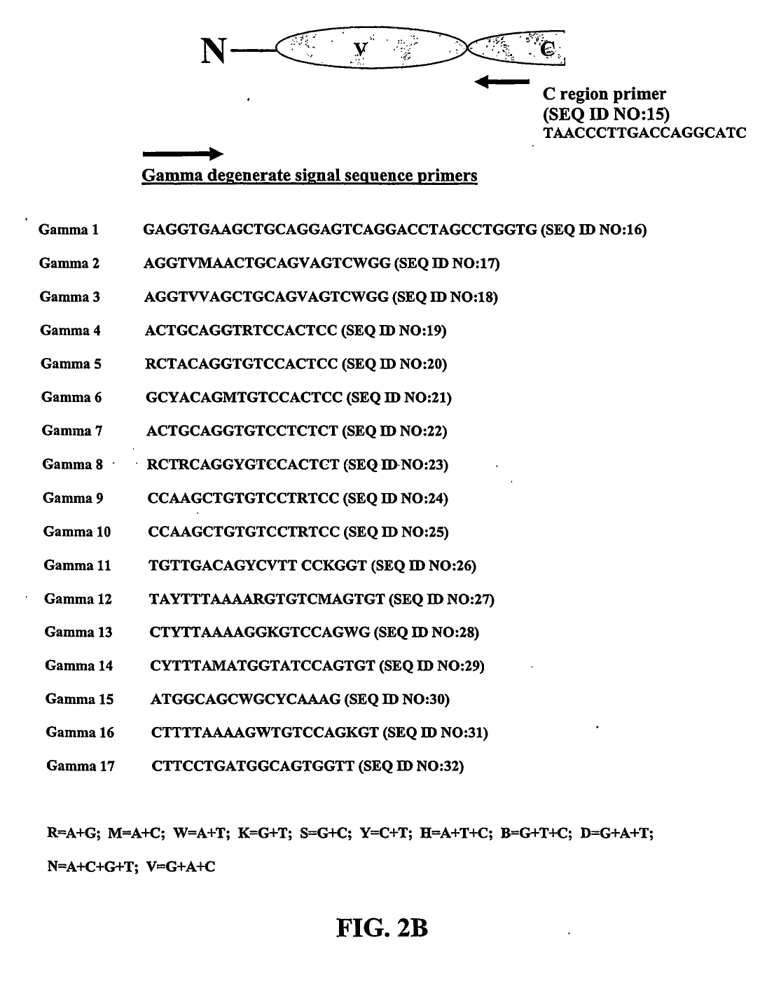

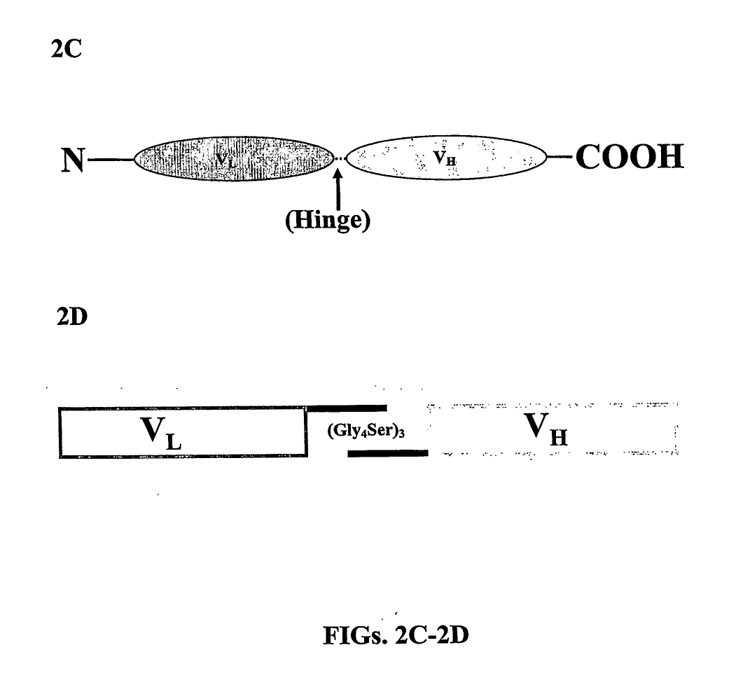

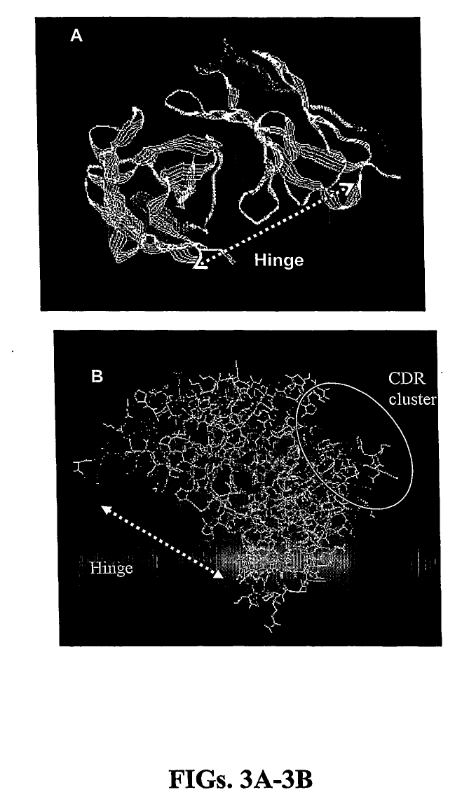

Generation of antibody single chain variable fragments (scFv). Single chain antibodies were constructed from four anti-gd secreting hybridomas, DL11, DL6, DL2 and 1D3. An additional scFv, directed against carcinoembryonic antigen (CEA) served as an independent control. Messenger RNAs from ˜5×10⁵-10⁶ hybridoma cells were isolated using Trizol (Invitrogen, CA) and cDNAs were generated by reverse transcription with Taq polymerase (‘Expand High Fidelity Taq polymerase’; Roche, Ind.). RT was primed with anti-sense oligonucleotides designed to anneal either to mouse kappa light chain or heavy chain constant region sequences, just downstream of the J-C junction (FIG. 2A-2D). Light and heavy chain hypervariable regions (V_{L} and V_{H}) were amplified by priming ‘sense’ PCR reaction products with panels of oligonucleotides (OGNs) designed from Kabat database sequences to be complementary to kappa (light chain) and gamma (heavy chain) signal or framework sequences (FIG. 3). In practice, pools of 11 degenerate OGN sequences were found to be sufficient to prime 100% of kappa chain reactions (14/14 hybridomas regardless of subclass). Similarly, a pool of 14 degenerate OGNs successfully amplified the gamma chains from these hybridomas. From each hybridoma, the resulting V_{L} and V_{H} cDNAs were sequenced and new specific primers were designed each of which included ⅔ of the fifteen amino acid (Gly₄Ser)₃ flexible hinge region, allowing the variable regions to be amplified and spliced together reconstituting the antigen binding site on reconformation (FIG. 2C-2D). To prevent complete overlap of the complementary hinge sequences, which would result in the introduction of a sub-optimal 10 amino acid (Gly₄Ser)₂ intervening segment, alternative glycine codons were used in each component of the hinge. Four of the scFvs were TA cloned into the bacterial expression vector pET101/D-TOPO (Invitrogen, Carlsbad, Calif.) which generates hexa-His tagged proteins after expression in vitro.

Expression of single chain antibodies in bacteria. Proteins were expressed in IPTG-induced E. Coli BL21 [DE3] (Invitrogen), released by sonication in PBS and inclusion bodies were separated by centrifugation. Proteins in inclusion bodies were solubulized with 6M guanidine HCl and purified by metal chelation. A stepwise dialysis procedure with addition of GSSG (oxidized glutathione; Sigma) and L-arginine in the final two steps was used to assist with in the formation of intra-chain disulphide bonds in order to optimize re-conformation and stability of the scFvs (Umetsu et al., 2003). Protein concentrations were measured using the BCA method (Pierce).

ELISA to quantafy binding of scFv to gD. Microtiter plate wells were coated with soluble gD (6 μg/ml) and then blocked with 1% skimmed milk. After incubation with serial two-fold dilutions of scFv, binding was detected with anti-V5, the alternative tag on the scFv, because the recombinant gD used in the assay was, like the single chain antibodies, tagged with hexa-His. Binding ratios were calculated in relation to an irrelevant (CEA-specific) scFv.

Virus growth, titration and plaque neutralization assays. HSV-1 (strain SC16) and HSV-2 (strain G) were grown and titrated in Vero cells as described. Titers were determined using a standard plaque assay (Russell, 1962). Cells were grown and maintained in Dulbecco modified Eagle medium supplemented with 10% (growth medium; GM) or 1% (maintenance medium; MM) fetal bovine serum. A plaque reduction assay was done in Vero cells to assess the neutralizing capabilities of each scFv. Briefly, 100-200 plaque forming units (PFU), diluted in MM, of either HSV-1 (strain SC16) or HSV-2 (strain G) were incubated at room temperature for 1 hour with serial ten-fold dilutions of each scFv in a total volume of 1 ml. After gentle shaking with 3×10⁶ Vero cells for a further 1 hour the samples were plated in 6 cm dishes (Nunc) in a total volume of 5 mls of GM containing 2% carboxymethylcellulose (CMC). Plaques were enumerated after 3 days incubation at 37° C. in a 5% CO₂ atmosphere.

Various purified, renatured bacterially expressed scFv were incubated in serial two-fold dilutions with 100 PFU HSV-1, strain SC16. 50% inhibition of plaque formation was achieved with 25 μg/ml of DL11-based scFv but no plaque reduction was observed with other scFv or an irrelevant scFv against carcinoembryonic antigen. Not only did DL11-scFv neutralize virus prior to infection but on some plates, exposed to lower concentrations, a reduction in plaque size was noted. After measuring the diameters of at least 100 plaques in cultures exposed to 25 μg/ml scFv, it was concluded that plaques were reduced in size by approximately ⅓, suggesting inhibition of cell-to-cell spread of virus.

Guinea pig model of GH. The microbicidal properties of scFv were tested using a guinea pig model of genital herpes. Female outbred Hartley guinea pigs weighing 350-400 grams were obtained from Charles River laboratories (Wilmington, Mass.). Prior to inoculation of each guinea pig with virus, the introitus was opened with a calcium alginate swab moistened in physiological saline and 1 ml of 1% CMC (vehicle) either alone or containing 500 mg of scFv was instilled using a pipette with a plastic tip. CMC was used as a vehicle to facilitate retention of the scFv in the vaginal vault. At various times thereafter, animals were challenged with 10⁶ PFU HSV-1 (strain SC16) or HSV-2 (strain G). Over the ensuing two weeks lesions were scored on a scale of 0-4 (0=no lesion; 1=erythema and swelling only; 2=small vesicles <2 mm; 3=coalescent or large vesicles >2 mm; 4=ulceration and maceration).

Results

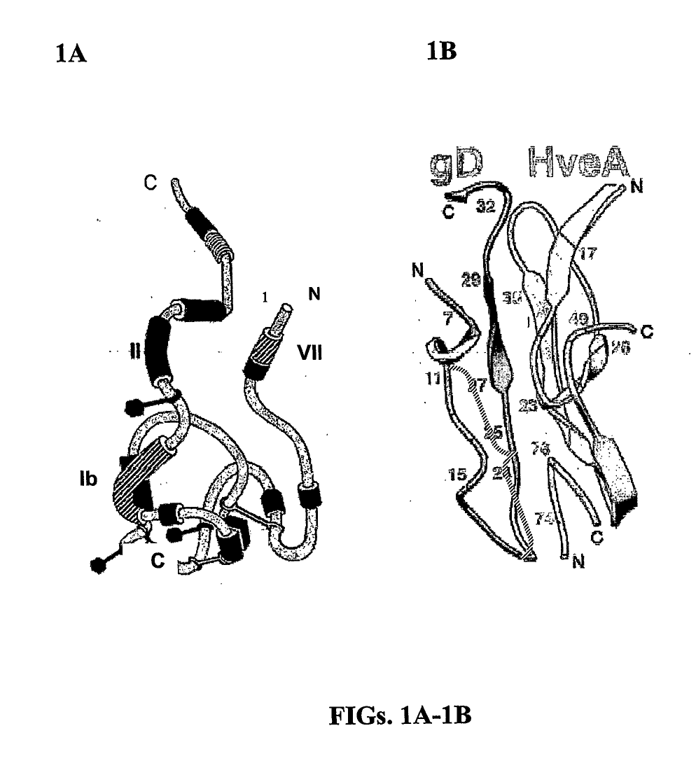

Construction and expression of single chain antibodies against gD. Four from the panel of anti-HSV gD hybridomas available were selected for scFv construction based on the known locations of their epitopes (Nicola et al., 1998) (FIG. 1A-1B) and knowledge about the neutralization properties of the antibodies produced by them. Of particular note are the properties of DL11, which neutralizes both HSV-1 and HSV-2 in the absence of complement and antibody binding to its conformational epitope is known to disrupt the interactions of gD both with Hve-A and nectin-1. Also 1D3 binds to a group VII neutralizing epitope that directly interferes with the interface between gD and HveA (FIG. 1B). A fifth scFv cassette, against CEA was excised from a plasmid encoding an anti-tumor chimeric T-cell receptor (T-body), kindly provided by Hinrich Abken (Cologne University, Germany). For production of cDNAs, individual V_{L} and V_{H} regions from each hybridoma were reverse transcribed using primers near the V_{H}-C_{H} and V_{L}-C_{L} junctions. For PCR cloning these primers were paired with a panel of degenerate primers derived from V_{H} or V_{L} signal sequences that were able to amplify the great majority of hybridoma heavy and light chains irrespective of antibody class or subclass (not shown). PCR products were sequenced directly to facilitate design of new primer sets allowing, on reamplification of hybridoma cDNAs, elimination of degenerate primer sequences introduced in the first reaction and introduction of ⅔ of a 15 amino acid hinge region comprising three repeats of four glycine and one serine residues (FIGS. 2C and 2D). V_{L} and V_{H} are not covalently linked in nature but flexible hinges of this design and length were shown previously to allow reconstruction of antibody binding sites when V_{L} and V_{H} are linked end-to-end (FIGS. 2D, 3A and 3B). Finally, the PCR products containing the overlapping hinge regions were ligated, PCR amplified and the resultant scFv cassette was TA cloned into pCR2.1TOPO. To generate the desired single chain antibodies, the cassettes were subcloned into the bacterial expression vector pET101-D.

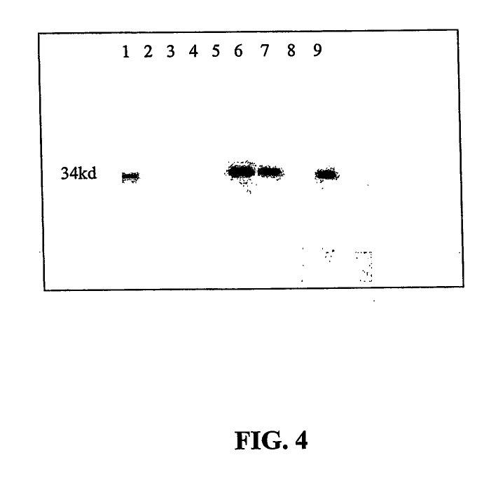

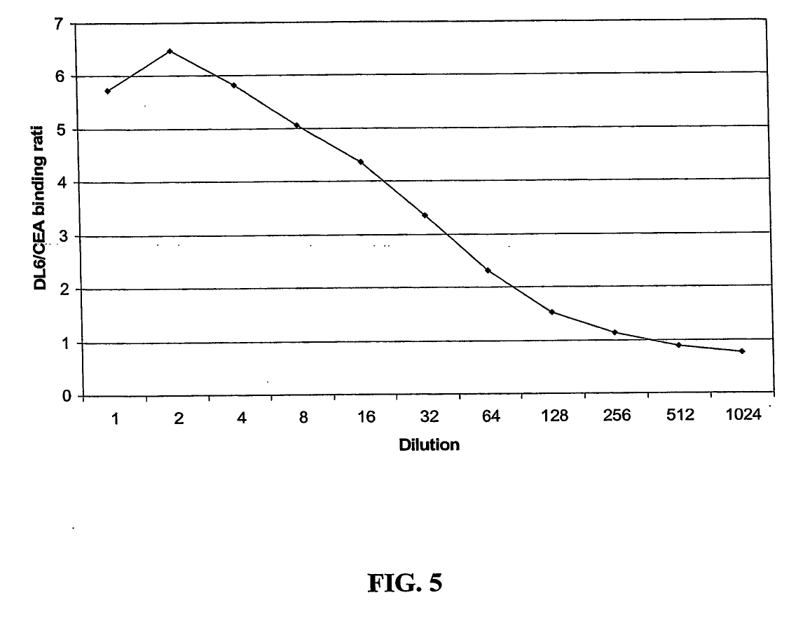

Bacterial expression and extraction of anti-gD single chain antibodies. The single chain antibodies were expressed in E. Coli strain BL21 using pET101-D (Invitrogen), which attaches hexa-His and V5 tags to expressed proteins for their isolation and identification. Bacteria were induced with IPTG, centrifuged and the supernatants tested for the presence of scFvs by western blotting using anti-His antibody (FIG. 4). Bacterial pellets were sonicated in phosphate buffered saline to release inclusion bodies and proteins were solubulized by addition of 6M guanidine (BL2 1). Nickel bead chelation was used to extract the His-tagged protein. Western blots of eluates from nickel beads (e.g., DL11 scFv from DL21; FIG. 4, lanes 6 and 7) identified scFvs that were released by this procedure. They were generally isolated at concentrations of 500-750 μg/ml from BL21. Re-folding and intra-chain disulphide bond formation were maximized by gradually reducing guanidine concentration by step-wise dialysis from 6M initially to 3M, then 2M, 1M, 0.5M and finally 0M, with addition of L-arginine and oxidized glutathione (GSSG) in final two steps (Umetsu et al., 2003). The ability of the single chain antibodies produced in this way to bind their target antigen was tested by determining their reaction with plastic bound gD by ELISA. Binding ratios were calculated in relation to the background binding of CEA scFv (e.g., DL6-based scFv; FIG. 5).

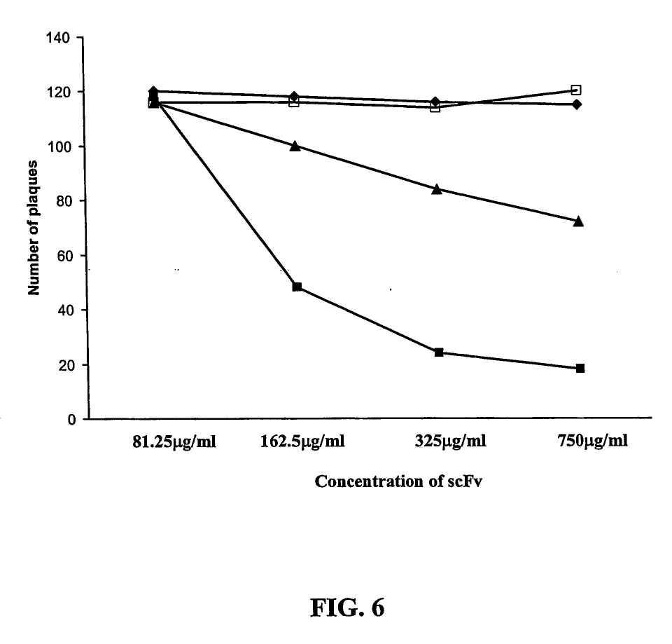

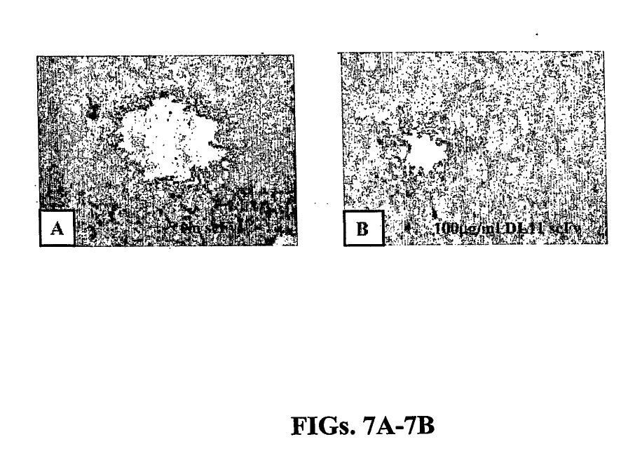

Selected anti-gD single chain antibodies neutralize HSV in vitro. The hypothesis that selected single chain antibodies can block infection of cells in vitro by reacting with an epitope that disrupts the interface between gD and HVEMs was tested by comparing the activities of the various bacterially expressed anti-gD scFv in a Vero cell-based plaque reduction assay. A scFv directed against an epitope on carcinoembryonic antigen was included as an irrelevant control. The results showed that DL11 and 1D3 scFvs inhibited plaque formation with decreasing efficiency. DL6 scFv showed minimal but reproducible activity (data not shown), whereas the other scFvs tested (DL2 and CEA) had no plaque reducing capability at all (FIG. 6). In addition to inhibition of plaque formation, when HSV-1 or HSV-2 strains were incubated with concentrations of DL1 scFv that were insufficient to completely inhibit plaque formation, the mean size of remaining plaques was significantly reduced for both viruses (e.g., FIGS. 7A and 7B; HSV-2). It was concluded that DL11 scFv could not only block infection of cells with HSV but also was able to inhibit cell-to-cell spread of virus.

Protection against HSV type 1 and type 2 genital herpes by administration of a DL11-based single chain antibody before virus. The HSV type-common and startling in vitro activities of single chain antibodies derived from hybridoma DL11 prompted the inventors to examine the ability of DL11 scFv to protect against vaginal HSV disease, using a well established guinea pig model of genital herpes (Stanberry et al., 1982, 1985). The vehicle selected for these preliminary studies was 1% carboxymethylcellulose because this is an inert compound that is used for its viscosity in our routine plaque assays.

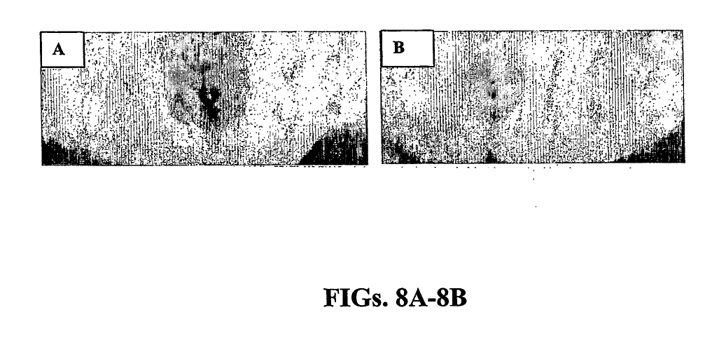

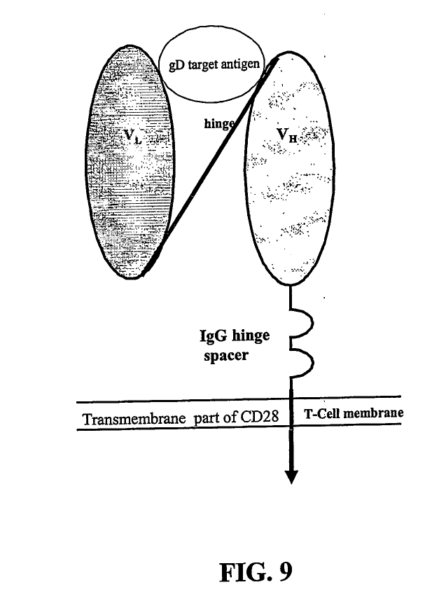

A pilot study was done with HSV-1, in which BL21 produced DL11 and DL2 single chain antibodies (0.5 mg/ml) were each instilled into the he vaginas of a guinea pig (0.1 ml/animal). Approximately 20 seconds later the guinea pigs were challenged with 5×10⁶ PFU HSV-1, strain SC16 and monitored for development and severity of primary disease. The result (FIG. 8A-8B) showed that DL11-based scFv completely protected the animal from lesion development whereas DL2-based scFv appeared to have, as expected, no effect.

Next a more ambitious test of microbicidal activity was attempted, using HSV-2 and a longer interval between scFv instillation and challenge. Two groups of 5 guinea pigs were each administered either DL11 or DL2 (control) scFv (1 ml/guinea pig). All animals were challenged with 10⁶ PFU of HSV-2, strain G and monitored daily as before. All except one animal were completely protected by DL11 scFv compared with controls which developed severe disease (Table 6).

TABLE 6
Protection of guinea pigs from HSV type 2 genital herpes by intravaginal
instillation of a DL11-based single chain antibody variable fragment
administered 20 minutes prior to challenge.

Lesion score* 5 days after infection*

| Animal | | Vehicle + 500 μg |
|---|---|---|
| number | Vehicle alone | DL11 scFv |
| 1 | 3 | 0 |
| 2 | 3 | 0 |
| 3 | 3-4 | 2 |
| 4 | 4 | 0 |
| 5 | 3 | 0 |

*Scale for assessment of lesions: 0 = no lesion; 1 = erythema and swelling only; 2 = small vesicles <2 mm; 3 = coalescent or large vesicles >2 mm; 4 = ulceration and maceration

### Example 2

Structural Modeling of scFvs

The 3-D structures of several scFvs were modeled using algorithms derived from the large number of antibodies that now have known sequences and structures. The models enabled the minimal sequences needed for expression of proteins with correctly conformed and aligned complementary determining regions (CDRs) (FIG. 3).

### Example 3

Chimeric T-Cell Receptors (T-Bodies)

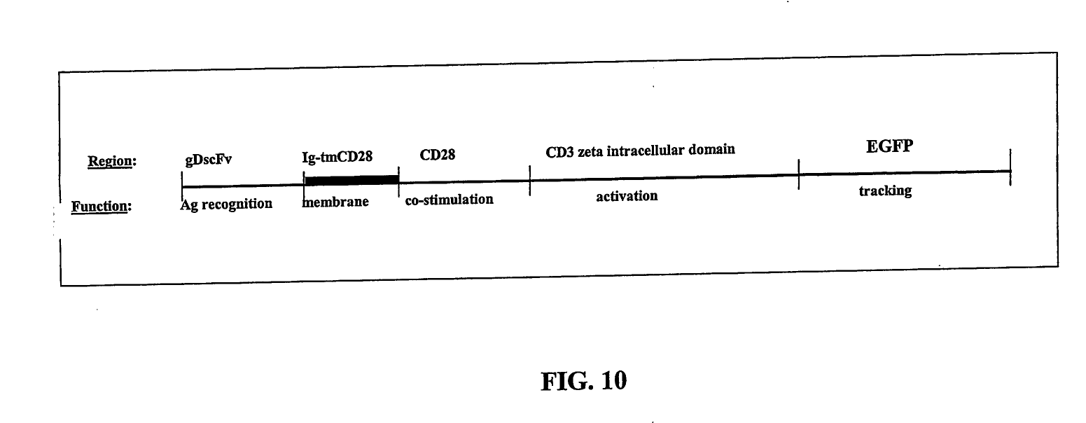

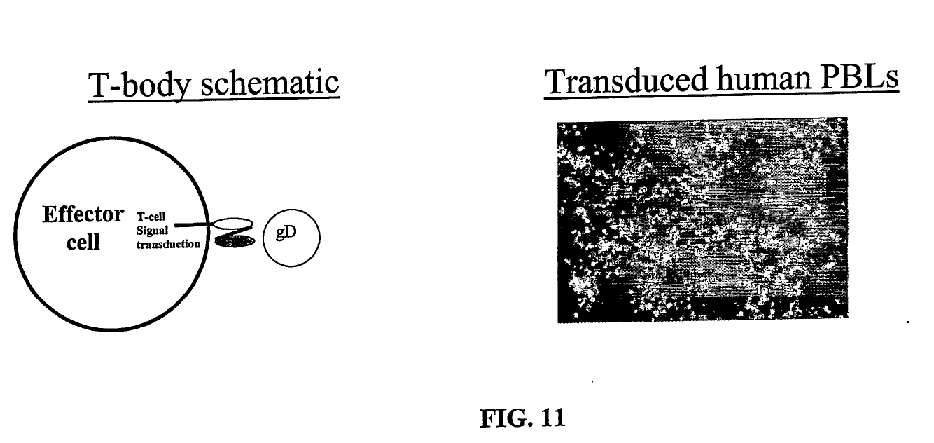

Structure of T-Body. FIG. 10 illustrates a T-body construct comprising an immunoglobulin spacer (Ig) and transmembrane (tmCD28) sequences. Alternative signaling domains were made and comprised human Ig FcR ITAM in place of CD3 zeta and also Syk. Insertion of EGFP driven by the same promoter allowed chTCR expression and T-body location to be monitored. (FIG. 10 and FIG. 11)

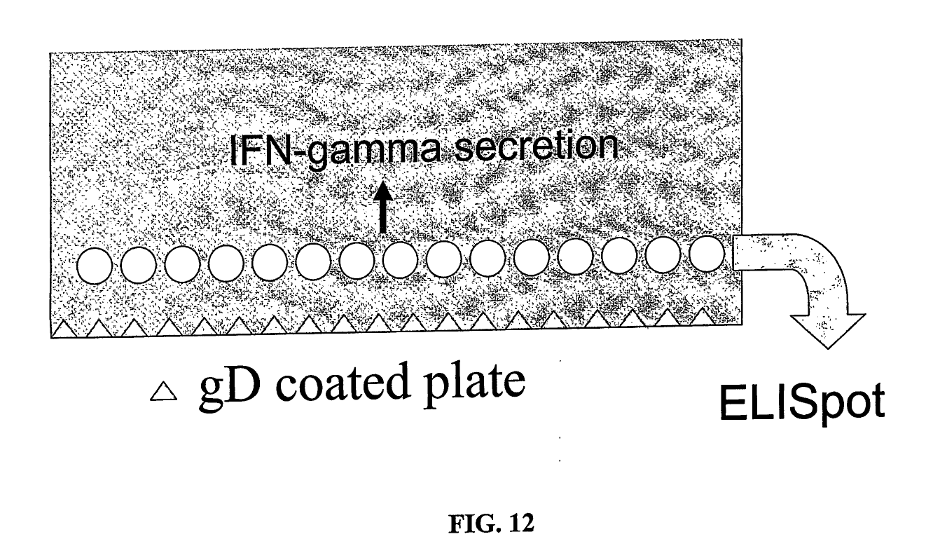

Generation of T-bodies. Host cells used: Human PBMCs, Jurkat, Mouse MD45 NK-like cells. Methods used: retroviral transduction, transient transfection (XtremeGENE Q2). Retroviral transduction was done using high titer virus (10⁶ PFU/ml) and three rounds of centrifugation (500 g) of virus and cells in Retronectin coated plates. (FIG. 12)

T-body reaction with gD in vitro. To demonstrate T-cell signaling by the chimeric receptor transduced Jurkat cells were used in the first instance. Synthesis and secretion of IFN-γ were used as physiologically relevant responses to indicate lymphocyte signaling by the chimeric receptor on contact with gD. (6 μg/ml) bound to plastic microtiter plate wells was used to test the responses of DL11-based T-bodies to their target epitope. An equivalent concentration of BSA was used as a control.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 3,817,837

U.S. Pat. No. 3,850,752

U.S. Pat. No. 3,939,350

U.S. Pat. No. 3,996,345

U.S. Pat. No. 4,275,149

U.S. Pat. No. 4,277,437

U.S. Pat. No. 4,366,241

U.S. Pat. No. 4,472,509

U.S. Pat. No. 4,554,101

U.S. Pat. No. 4,659,774

U.S. Pat. No. 4,682,195

U.S. Pat. No. 4,683,202

U.S. Pat. No. 4,684,611

U.S. Pat. No. 4,797,368

U.S. Pat. No. 4,816,571

U.S. Pat. No. 4,879,236

U.S. Pat. No. 4,946,778

U.S. Pat. No. 4,952,500

U.S. Pat. No. 4,959,463

U.S. Pat. No. 5,021,236

U.S. Pat. No. 5,139,941

U.S. Pat. No. 5,141,813

U.S. Pat. No. 5,258,498

U.S. Pat. No. 5,264,566

U.S. Pat. No. 5,322,783

U.S. Pat. No. 5,359,681

U.S. Pat. No. 5,384,253

U.S. Pat. No. 5,428,148

U.S. Pat. No. 5,532,210

U.S. Pat. No. 5,538,877

U.S. Pat. No. 5,538,880

U.S. Pat. No. 5,550,318

U.S. Pat. No. 5,554,744

U.S. Pat. No. 5,563,055

U.S. Pat. No. 5,574,146

U.S. Pat. No. 5,580,859

U.S. Pat. No. 5,589,466

U.S. Pat. No. 5,591,616

U.S. Pat. No. 5,602,244

U.S. Pat. No. 5,610,042

U.S. Pat. No. 5,645,897

U.S. Pat. No. 5,656,610

U.S. Pat. No. 5,658,727

U.S. Pat. No. 5,667,988

U.S. Pat. No. 5,702,932

U.S. Pat. No. 5,705,629

U.S. Pat. No. 5,736,524

U.S. Pat. No. 5,780,448

U.S. Pat. No. 5,789,215

U.S. Pat. No. 5,840,300

U.S. Pat. No. 5,871,986

U.S. Pat. No. 5,932,448

U.S. Pat. No. 5,945,100

U.S. Pat. No. 5,981,274

U.S. Pat. No. 5,994,136

U.S. Pat. No. 5,994,624

U.S. Pat. No. 6,013,516

U.S. Pat. No. 6,129,914

U.S. Pat. No. 6,133,426

Anadrade et al., J. Biochem (Tokyo), 128:891-895,2000.

Archer et al., Antonie Van Leeuvenhoek, 65:245-250, 1994.

Ausubel et al., In: Current Protocols in Molecular Biology, John, Wiley & Sons, Inc, New York, 1994.

Baichwal and Sugden, In: Gene Transfer, Kucherlapati (Ed.), NY, Plenum Press, 117-148, 1986.

Bittner et al., Methods in Enzyinol, 153:516-544, 1987.

Blomer et al., J. Virol., 71(9):6641-6649, 1997.

Blundell et al., Nature, 326:347-352, 1987.

Brutlag et al., Comput. Appl. Biosci., 6(3):237-245, 1990.

Chen and Okayama, Mol. Cell Biol., 7(8):2745-2752, 1987.

Chou and Fasman, “Prediction of protein conformation,” Biochemistry, 13(2):222-245, 1974.

Chou and Fasman, Adv. Enzymol., 47:45-148, 1978.

Chou and Fasman, Annu. Rev. Biochem., 47:251-276, 1978.

Chou and Fasman, Biochemistry, 13(2):211-222, 1974.

Chou and Fasman, Biophys. J., 26(3):385-399, 1979.

Colcher etal., J. Natl. Cancer Inst., 82:1191, 1990.

Conrath et al., J. Biol. Chem., 276-7346-7350, 2001.

Cotten et al., Proc. Natl. Acad. Sci. USA, 89(13):6094-6098, 1992.

Coupar et al., Gene, 68:1-10, 1988.

Curiel, Nat. Immun., 13(2-3):141-164, 1994.

Davis et al., Biotechnology, 9:165-169, 1991.

Desmyter et al., J. Biol. Chem., 276:26285-26290, 2001.

Durand et al, Enzyme Microb. Technol., 6:341-346, 1988.

Eldin et al., J. Immunol. Methods, 201:67-75, 1997.

Elias and Heise, 8:1-9, 1994.

European Pat Appln. EP 266 032

Fechheimer et al., Proc. Natl. Acad. Sci. USA, 84:8463-8467, 1987.

Fetrow and Byrant, Biotechnology, 11(4):479-484, 1993.

Fraley et al., Proc. Natl. Acad. Sci. USA, 76:3348-3352, 1979.

Freyre et al., J. Biotechnol., 76:157-163, 2000.

Friedmann, Science, 244:1275-1281, 1989.

Froehler et al., Nucleic Acids Res., 14(13):5399-5407, 1986.

Glorioso et al., Mol. Biotechnol., 4(1):87-99, 1995.

Gopal, Mol. Cell Biol., 5:1188-1190, 1985.

Gouka et al., Appl. Microbiol. Biotechnol., 47:1-11, 1997.

Graham and Van Der Eb, Virology, 52:456-467, 1973.

Grunhaus et al., Seminar in Virology, 200(2):535-546, 1992.

Harlan and Weintraub, J. Cell Biol., 101:1094-1099, 1985.

Harlow and Lane, Antibodies: A Laboratory Manual. Cold Spring Harbor Laboratory Press, Cold Spring harbor, N.Y., 553-612, 1988.

Herz and Roizman, Cell, 33(1):145-155, 1983.

Horwich et al. J. Virol., 64:642-650, 1990.

Horwitz et al., Proc. Natl. Acad. Sci. USA, 85:8678-8682, 1988.

Jameson and Wolf, Comput. Appl. Biosci., 4(1):181-186, 1988.

Kaeppler et al., Plant Cell Reports, 9:415-418, 1990.

Kaneda et al., Science, 243:375-378, 1989.

Kato et al, J. Biol. Chem., 266:3361-3364, 1991.

Kelleher and Vos, Biotechniques, 17(6):1110-7, 1994.

Keranen et al., Curr. Opin. Biotechnol., 6:534-537, 1995.

Kostelney et al., J. Imnmunol., 148:1547-1553, 1992.

Laughlin et al., J. Virol., 60(2):515-524, 1986.

Lebkowski et al., Mol. Cell. Biol., 8(10):3988-3996, 1988.

Lifson, Lancet., 343:1306-1307, 1994.

Louv et al., J. Infect. Dis., 158:518-523, 1988.

Mack, Proc. Natl. Acad Sci. USA, 92:7021-7025, 1995.

Malecki et al., Proc. Natl. Acad. Sci. USA, 99:213-218, 2002.

Mandell, Douglas and Bennett's Principles and Practice of Infectious Diseases, 5^{th} Ed., Churchill Livingstone, Inc., September. 1998.

Mann et al., Cell, 33:153-159, 1983.

McGeoch et al., J. Mol. Biol., 181:1, 1985.

McGeoch et al., Nucleic Acids Res., 14:1727, 1986.

McGeoch et al., J. Gen. Virol. 69: 1531, 1988.

McLaughlin et al., J. Virol., 62(6):1963-1973, 1988.

Milenic et al., Cancer Res., 51:6363-6371, 1991.

Miller et al., Am. J. Clin. Oncol., 15(3):216-221, 1992.

Muzyczka, Curr. Topics Microbiol. Immunol., 158:97-129, 1992.

Nabel et al., Science, 244(4910):1342-1344, 1989.

Naldini et al., Science, 272(5259):263-267, 1996.

Nevalainen et al., J. Biotechnol., 37:193-200, 1994.

Nicolas and Rubinstein, In: Vectors: A survey of molecular cloning vectors and their uses, Rodriguez and Denhardt (Eds.), Stoneham: Butterworth, 494-513, 1988.

Nicolau and Sene, Biochim. Biophys. Acta, 721:185-190, 1982.

Nicolau et al., Methods Enzymol., 149:157-176, 1987.

Nyyssonen et al., Biotechnology, 11:591-595, 1993.

Omirulleh et al., Plant Mol. Biol., 21(3):415-28, 1993.

Paskind et al., Virology, 67:242-248, 1975.

PCT Appln. WO 92/01797

PCT Appln. WO 94/09699

PCT Appln. WO 94/12520

PCT Appln. WO 95/06128

Pennell et al., Res. Immunol., 149:599-603, 1998.

Perry and McGeoch, J. Gen. Virol., 69:2831, 1988.

Pierce Immunotechnology catalog and handbook, A12-A13, 1991.

Potrykus et al., Mol. Gen. Genet., 199:183-188, 1985.

Potter et al., Proc. Natl. Acad. Sci. USA, 81:7161-7165, 1984.

Punt et al., Trends Biotechnol., 20:200-206, 2002.

Radzio et al., Process-Biochem., 32:529-539, 1997.

Ridder et al., Biotechnology, 13:255-260, 1995.

Ridgeway, In: Vectors: A survey of molecular cloning vectors and their uses, Rodriguez and Denhardt (Eds.), Stonehamn:Butterworth, 467-492, 1988.

Rippe et al., Mol. Cell Biol., 10:689-695, 1990.

Rosenberg et al., Sex. Trans. Dis., 20:41-44, 1993.

Roux et al., Proc. Natl. Acad. Sci. USA, 86:9079-9083, 1989.

Russell, Nature, 195:1028-1029 1962.

Sackett et al., In: Clinical Epidemilogy: A Basic Science for Clinial Medicine, Little Brown and Co, 106-107, 1985.

Sambrook et al., In: Molecular cloning, Cold Spring Harbor Laboratory Press, Cold Spring Harbor, N.Y., 2001.

Sexually Transmitted Diseases in Obstetrics and Gynecology, Faro (Ed.), Lippincott Williams & Williams, June, 2001.

Sexually Transmitted Diseases, Holmes, Mardh, Wasserheit (Eds.), McGraw-Hill, January, 1999.

Sexually Transmitted Diseases, Vol. 5 Mandell and Rein (Eds.), Churchill Livingstone, Inc., January. 1996.

Stanberry et al., J. Infect. Dis., 146(3):397-404, 1982.

Stanberry et al., Virol., 55(2):322-328 1985.

Swennen et al., Microbiology, 148:41-50, 2002.

Temin, In: Gene Transfer, Kucherlapati (Ed.), NY, Plenum Press, 149-188, 1986.

Tratschin et al., Mol. Cell. Biol., 4:2072-2081, 1984.

Tur-Kaspa et al., Mol. Cell Biol., 6:716-718, 1986.

Umetsu et al., J. Biol. Chem., 14;278(11):8979-8987, 2003

Verdoes et al., Appl. Microbiol. Biotechnol., 43:195-205, 1995.

Ward et al., Biotechnology, 8:435-440, 1990.

Weinberger et al., Science, 228:740-742, 1985.

Wilson et al., Science, 244:1344-1346, 1989.

Wolf et al., Comput. Appl. Biosci., 4(1):187-191, 1988.

Wong et al., Gene, 10:87-94, 1980.

Wu and Wu, Biochemistry, 27:887-892, 1988.

Wu and Wu, J. Biol. Chein., 262:4429-4432, 1987.

Yokota et al., Cancer Res., 52:3402, 1992.

Zekeng et al., AIDS, 7:725-731, 1993.

Zufferey et al., Nat. Biotechnol., 15:871-875, 1997.

1. A single chain antibody that specifically binds to an HSV glycoprotein.

2. The single chain antibody of claim 1, wherein the HSV glycoprotein is HSV glycoprotein D (HSV gD).

3. The single chain antibody of claim 1, further comprising a transmembrane region of a cell surface receptor.

4. The single chain antibody of claim 3, wherein the cell surface receptor is a T-cell receptor.

5. The single chain antibody of claim 1, wherein the single chain antibody binds site VII or site Ib of HSV gD.

6. The single chain antibody of claim 1, coupled to a second antibody that binds to an HSV glycoprotein or other pathogen associated protein.

7. The single chain antibody of claim 6, wherein the antibody is a bi-specific antibody.

8. An isolated polynucleotide comprising a nucleic acid sequence encoding a single chain antibody that specifically binds a HSV glycoprotein.

9. The isolated polynucleotide of claim 8, wherein the HSV glycoprotein is a HSV glycoprotein D protein (HSV gD).

10. The isolated polynucleotide of claim 9, wherein the nucleic acid sequence is comprised in an expression cassette.

11. The isolated polynucleotide of claim 10, wherein the expression cassette further comprises an HSV promoter.

12. A composition comprising the single chain antibody of claim 1.

13. The composition of claim 12, further comprising at least a second single chain antibody with a binding affinity for a pathogenic microbe that causes a sexually transmitted disease.

14. The composition of claim 13, wherein binding of the the second single chain antibody to the microbe reduces the infectivity of the microbe.

15. The composition of claim 14, wherein the microbe is HIV, HSV, chlamydia, or Hepatitis B virus.

16. The composition of claim 12, wherein the composition is comprised in a pharmaceutically acceptable composition.

17. The composition of claim 12, further comprising an antiviral therapeutic agent.

18. The composition of claim 17, wherein the antiviral therapeutic agent is a nucleoside analog.

19. The composition of claim 16, wherein the pharmaceutically acceptable composition is a topical composition.

20. The composition of claim 19, wherein the topical composition is a foam or gel.

21. (canceled)

22. The composition of claim 1, further comprising at least a second antibody.

23.-27. (canceled)

28. A method of producing an HSV single chain antibody comprising:
a) introducing into a cell an expression cassette encoding the single chain antibody of claim 1; and
b) isolating the single chain antibody expressed by the cell.

29. The method of claim 28, wherein isolating the single chain antibody comprises purifying the single chain antibody.

30. The method of claim 29, wherein purifying the single chain antibody comprises affinity purification.

31.-35. (canceled)

36. A method of preventing or treating an HSV infection comprising administering to a subject a pharmaceutically acceptable composition comprising at least a first single chain antibody that specifically binds a HSV glycoprotein.

37. The method of claim 36, wherein the first single chain antibody binds an epitope in a HSV glycoprotein.

38. The method of claim 37, wherein the HSV glycoprotein is HSV gD.

39. The method of claim 36, further comprising determining the subject was exposed to HSV.

40. The method of claim 36, wherein the proteinaceous composition further comprises at least a second single chain antibody having a binding specificity for at least a second microbe.

41. The method of claim 40, wherein at least a second microbe is HSV, HIV, chlamydia, or HepB.

42.-45. (canceled)

46. A method for determining the presence of HSV in a sample suspected of containing HSV, the method comprising exposing the sample to a single chain antibody that binds an HSV glycoprotein.

47. The method of claim 46, wherein the HSV glycoprotein is HSV gD.


SEQUENCE LISTING (36 sequences)

SEQ ID NO: 1 - DNA, 921 bp, Artificial Sequence
  Description of Artificial Sequence  Synthetic Primer
atggagacagacacactcctgctatgggtactgctgctctgggttccaggttccactggtgacgcggcccagccggccaggcgcgcgcgccgtacgaagcttggtaccgagctcggatccgacatccagatgacccagtctccatcctccttatctgcctctctgggagaaagagtcagtctcacttgtcgggcaagtcaggaaattagtgcttacttaagctggcttcagcagaaaccagatggaactattaaacgcctgatctacgccgcatccactttagattctggtgtcccaaaaaggttcagtggcagtaggtctgggtcagattattctctcaccatcagcagccttgagtctgaagattttgcagactattactgtctacaatatgctagttatccgctcacgttcggtgctgggaccaagctggagctgggcggtggcggatcgggtggaggaggcagcggaggcggtggttcggtgaagctgcaggagtctggacctgagctggtgaagcctggggcttcagtgaagatgtcctgcaaggcttctggctacaccttcacaagctcctatgtacactgggtgaagcagaggcctggacagggacttgagtggattggatggatttatcctggacatagtagtactaagtacaatgagaagttcatgggcaagaccatattgactgcggacaaatcctccagcacagcctacatgttgctcagcagcctgacctctgaggactctgcgatatatttctgtacaaggcaggaggtacgactctggtacttcgatgtctggggcgcagggaccacggtcaccgtctccgcggccgctcgaggagggcccgaacaaaaactcatctcagaagaggatctgaatagcgccgtcgaccatcatcatcatcatcattga

SEQ ID NO: 2 - protein, 306 aa, Artificial Sequence
  Description of Artificial Sequence  Synthetic Peptide
METDTLLLWVLLLWVPGSTGDAAQPARRARRTKLGTELGSDIQMTQSPSSLSASLGERVSLTCRASQEISAYLSWLQQKPDGTIKRLIYAASTLDSGVPKRFSGSRSGSDYSLTISSLESEDFADYYCLQYASYPLTFGAGTKLELGGGGSGGGGSGGGGSVKLQESGPELVKPGASVKMSCKASGYTFTSSYVHWVKQRPGQGLEWIGWIYPGHSSTKYNEKFMGKTILTADKSSSTAYMLLSSLTSEDSAIYFCTRQEVRLWYFDVWGAGTTVTVSAAARGGPEQKLISEEDLNSAVDHHHHHH

SEQ ID NO: 3 - DNA, 15 bp, Artificial Sequence
  Description of Artificial Sequence  Synthetic Primer
tggtgggaagatgga

SEQ ID NO: 4 - DNA, 32 bp, Artificial Sequence
  Description of Artificial Sequence  Synthetic Primer
ggtgatatcgtgatracmcargatgaactctc

SEQ ID NO: 5 - DNA, 32 bp, Artificial Sequence
  Description of Artificial Sequence  Synthetic Primer
ggtgatatcwtgmtgacccaawctccactctc

SEQ ID NO: 6 - DNA, 32 bp, Artificial Sequence
  Description of Artificial Sequence  Synthetic Primer
ggtgatatcgtkctcacycartctccagcaat

SEQ ID NO: 7 - DNA, 19 bp, Artificial Sequence
  Description of Artificial Sequence  Synthetic Primer
ctgwtgttctggattcctg

SEQ ID NO: 8 - DNA, 18 bp, Artificial Sequence
  Description of Artificial Sequence  Synthetic Primer
gtgctctggattcgggaa

SEQ ID NO: 9 - DNA, 21 bp, Artificial Sequence
  Description of Artificial Sequence  Synthetic Primer
tcagcttcytgctaatcagtg

SEQ ID NO: 10 - DNA, 19 bp, Artificial Sequence
  Description of Artificial Sequence  Synthetic Primer
tgggtatctggtrcstgtg

SEQ ID NO: 11 - DNA, 19 bp, Artificial Sequence
  Description of Artificial Sequence  Synthetic Primer
gtttcmaggtrccagatgt

SEQ ID NO: 12 - DNA, 21 bp, Artificial Sequence
  Description of Artificial Sequence  Synthetic Primer
tgttttcaaggtrccagatgt

SEQ ID NO: 13 - DNA, 20 bp, Artificial Sequence
  Description of Artificial Sequence  Synthetic Primer
ctstggttgtctggtgttga

SEQ ID NO: 14 - DNA, 18 bp, Artificial Sequence
  Description of Artificial Sequence  Synthetic Primer
tgctkckctgggttccag

SEQ ID NO: 15 - DNA, 20 bp, Artificial Sequence
  Description of Artificial Sequence  Synthetic Primer
taacccttgaccaggcatcc

SEQ ID NO: 16 - DNA, 36 bp, Artificial Sequence
  Description of Artificial Sequence  Synthetic Primer
gaggtgaagctgcaggagtcaggacctagcctggtg

SEQ ID NO: 17 - DNA, 22 bp, Artificial Sequence
  Description of Artificial Sequence  Synthetic Primer
aggtvmaactgcagvagtcwgg

SEQ ID NO: 18 - DNA, 22 bp, Artificial Sequence
  Description of Artificial Sequence  Synthetic Primer
aggtvvagctgcagvagtcwgg

SEQ ID NO: 19 - DNA, 18 bp, Artificial Sequence
  Description of Artificial Sequence  Synthetic Primer
actgcaggtrtccactcc

SEQ ID NO: 20 - DNA, 18 bp, Artificial Sequence
  Description of Artificial Sequence  Synthetic Primer
rctacaggtgtccactcc

SEQ ID NO: 21 - DNA, 18 bp, Artificial Sequence
  Description of Artificial Sequence  Synthetic Primer
gcyacagmtgtccactcc

SEQ ID NO: 22 - DNA, 18 bp, Artificial Sequence
  Description of Artificial Sequence  Synthetic Primer
actgcaggtgtcctctct

SEQ ID NO: 23 - DNA, 18 bp, Artificial Sequence
  Description of Artificial Sequence  Synthetic Primer
rctrcaggygtccactct

SEQ ID NO: 24 - DNA, 18 bp, Artificial Sequence
  Description of Artificial Sequence  Synthetic Primer
ccaagctgtgtcctrtcc

SEQ ID NO: 25 - DNA, 18 bp, Artificial Sequence
  Description of Artificial Sequence  Synthetic Primer
ccaagctgtgtcctrtcc

SEQ ID NO: 26 - DNA, 20 bp, Artificial Sequence
  Description of Artificial Sequence  Synthetic Primer
tgttgacagycvttcckggt

SEQ ID NO: 27 - DNA, 22 bp, Artificial Sequence
  Description of Artificial Sequence  Synthetic Primer
taytttaaaargtgtcmagtgt

SEQ ID NO: 28 - DNA, 20 bp, Artificial Sequence
  Description of Artificial Sequence  Synthetic Primer
ctyttaaaaggkgtccagwg

SEQ ID NO: 29 - DNA, 21 bp, Artificial Sequence
  Description of Artificial Sequence  Synthetic Primer
cytttamatggtatccagtgt

SEQ ID NO: 30 - DNA, 17 bp, Artificial Sequence
  Description of Artificial Sequence  Synthetic Primer
atggcagcwgcycaaag

SEQ ID NO: 31 - DNA, 21 bp, Artificial Sequence
  Description of Artificial Sequence  Synthetic Primer
cttttaaaagwtgtccagkgt

SEQ ID NO: 32 - DNA, 19 bp, Artificial Sequence
  Description of Artificial Sequence  Synthetic Primer
cttcctgatggcagtggtt

SEQ ID NO: 33 - protein, 904 aa, human herpesvirus 1
MRQGAPARGRRWFVVWALLGLTLGVLVASAAPSSPGTPGVAAATQAANGGPATPAPPAPGAPPTGDPKPKKNRKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 34 - protein, 904 aa, human herpesvirus 2
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINTKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 35 - protein, 394 aa, human herpesvirus 1
MGGAAARLGAVILFVVIVGLHGVRSKYALVDASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRHTQKAPKRIRLPHIREDDQPSSHQPLFY

SEQ ID NO: 36 - protein, 393 aa, human herpesvirus 2
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY